(19) United States
(12) Patent Application Publication
(10) Pub. No.: US 2024/0331839 A1
(43) Pub. Date: Oct. 3, 2024

(54) DYNAMICALLY MODELING THE EFFECT OF FOOD ITEMS AND ACTIVITY ON A PATIENT'S METABOLIC HEALTH

(71) Applicant: Twin Health, Inc., Mountain View, CA (US)

(72) Inventors: Javad SOVIZI, San Ramon, CA (US); James WILSON, Sunnyvale, CA (US); Frederick HADLEY, Sunnyvale, CA (US); Christopher SZETO, Mountain View, CA (US); Terrence Chun Yin POON, Foster City, CA (US)

(21) Appl. No.: 18/623,790

(22) Filed: Apr. 1, 2024

Related U.S. Application Data
(60) Provisional application No. 63/493,424, filed on Mar. 31, 2023.

(51) Int. Cl. G16H 20/60; G16H 40/67; G16H 50/20; G16H 50/70

(52) U.S. Cl. CPC G16H 20/60; G16H 40/67; G16H 50/20; G16H 50/70

(57) ABSTRACT

Disclosed herein is a method, system, and computer-readable medium for recommending foods to a patient. The disclosure includes accessing a record of food items recorded by a patient, including a classification of each food item. The method retrieves a current metabolic profile of the patient. Using a machine learning model, the method determines an updated classification for the food items and generates a notification for the patient. Additionally disclosed is a method, system, and computer-readable medium for recommending activities and activity times to a patient. The method includes accessing a record of activities previously recorded by a patient, each entry in the recordings including a duration of the activity and biosignal measurements. The method determines an effect of each activity on the metabolic state of the patient using a machine learning model. The method identifies activities that improve the patient's metabolic state and generates a recommendation for the patient.

### CROSS REFERENCE TO RELATED APPLICATIONS

This application claims the benefit of U.S. Provisional Application No. 63/493,424, filed on Mar. 31, 2023, which is incorporated by reference in its entirety.

### BACKGROUND

### Field of Art

The disclosure relates generally to a patient health management platform, and more specifically, to a personalized treatment platform for virtually monitoring the effects of food items and activities on a metabolic state of a patient using machine-learning models.

### Description of the Related Art

Metabolic dysfunction, for example the metabolic dysfunction that occurs in type 2 diabetes, hypertension, lipid problems, heart disease, non-alcoholic fatty liver disease, polycystic ovarian syndrome, cancer, and dementia, is a major contributor to health care costs. Conventional disease management platforms or techniques either ignore or fail to fully understand important markers, such as blood sugar dysregulation, and root causes for these diseases, such as processed foods and a lack of exercise. Traditionally, these platforms are designed to treat symptoms as they arise rather than treating the root cause of the disease—the deterioration of a patient's metabolic health.

Additionally, every person is unique in their metabolic health. Because each person's underlying metabolic health is unique, the same foods or activities may have different effects on the metabolic health of different patients. For example, a given food may improve the metabolic health of one patient while worsening the metabolic health of another. As another example, a given physical activity may improve the metabolic health of one patient while worsening the metabolic health of another. However, conventional disease management platforms are incapable of generating patient-specific recommendations for improving metabolic health by consuming foods or participating in activities.

### SUMMARY

A patient health management platform for managing a patient's metabolic diseases generates insight into the effects of food items and activities on the patient's metabolism using machine learning techniques. The platform establishes a personalized metabolic profile for a patient based on biosignals collected for the patient, for example blood glucose measurements. These biosignals are input to machine-learning models personalized for the patient to determine patient-specific insights into the effects of different food items and activities on their metabolic health. The patient health management platform implements a first machine-learning model trained to predict a patient's metabolic response to particular food items and a second machine-learning model trained to predict the patient's metabolic response to particular physical activities. Based on the predictions generated by the machine-learning models, the patient health management platform generates personalized recommendations for the patient to improve their metabolic health by consuming or avoiding particular foods, participating in particular activities, or both. The patient health management platform may further identify a subset of food items whose classification has changed between a preceding time period and a current time period. The recommendation may also provide insights and instructions for the patient to perform particular activities at specific times of the day to improve the patient's metabolic health.

In one embodiment, the patient health management platform implements a machine-learning model trained to classify food items based on the effect of the food item on a metabolic state of the patient. The patient health management platform accesses a record of food items recorded by the patient. Each recorded food item includes a current classification describing the effect of the food item on a metabolic state of the patient. The patient health management platform retrieves a current metabolic profile of the patient, which includes biosignal measurements collected during a current time period by one or more wearable sensors worn by the patient and a current metabolic state of the patient determined for the current time period. The patient health management platform encodes the record of food items, the biosignal measurements of the metabolic profile, and the current metabolic state of the patient into a vector representation.

The patient health management platform determines an updated classification for each food item of the record of food items by inputting the vector representation to a patient-specific metabolic food model. The metabolic food model is iteratively trained to classify food items based on a training dataset of previously classified food items labeled with a corresponding effect of the previously classified food item on a metabolic state of a patient. The patient health management platform identifies a subset of the recorded food items where the updated classification differs from the current classification and generates a notification for display to the patient via an application on a computing device. The notification may include a graphic representation of the identified subset for display to the patient.

In one embodiment, the patient health management platform implements a machine-learning model trained to classify activities based on the effect of the food item on a metabolic state of the patient. The patient health management platform accesses a record of activities for the patient. Each recorded activity includes a duration of the activity and biosignal measurements collected for the patient during the activity. The patient health management platform retrieves a current metabolic profile of the patient, which includes biosignal measurements collected during a current time period by one or more wearable sensors worn by the patient and a current metabolic state of the patient determined for the current time period. The patient health management platform encodes the record of activities, the biosignal measurements of the metabolic profile, and the current metabolic state of the patient into a vector representation.

The patient health management platform determines an updated classification for each food item of the record of activities by inputting the vector representation to a patient-specific metabolic activity model. The metabolic activity model is iteratively trained to predict the effect of an activity on a patient's metabolic state based on a training dataset of previously recorded activities labeled with a corresponding effect on a metabolic state of a patient. The patient health management platform identifies a subset of activities that improve the metabolic state of the patient based on the effects determined by the metabolic activity model and generates a notification with a graphic representation of the identified subset of activities for display to the patient via an application on a computing device.

This disclosure provides a personalized metabolic state program that provides tailored nutrition and exercise recommendations aligned with individual preferences. The disclosed method monitors and classifies food items for evaluating and managing a patient's metabolic state. Every patient has unique dietary preferences, cultural backgrounds, and metabolic needs. By tracking the food intake and monitoring the biosignals, the method understands the food intake timing, frequency, and composition, which enables personalized dietary recommendations, enhancing adherence and compliance with dietary modifications. Similarly, the disclosed method monitors physical activities for assessing and managing a patient's metabolic state. Regular activities contribute to energy expenditure, supporting weight management and metabolic balance. By assessing the frequency, intensity, and duration of exercise, the disclosed method may provide valuable insights into exercise habits, tailor exercise prescriptions to meet individual patient needs and goals.

Moreover, this disclosure uses machine learning models to monitor and determine a patient's metabolic state, classify food item and physical activities, and provide recommendations, etc. Machine learning models analyze vast amounts of data, including food intake, physical activities, biosignals, and health data records, to generate personalized recommendations tailored to individual patients. By considering multiple variables and their interactions, machine learning models disclosed herein provide more nuanced and personalized insights into metabolic health than traditional approaches. Additionally, the disclosed machine learning models integrate data from various sources, including wearable devices, electronic health records, genetic information, and lifestyle data, to provide a comprehensive view of a patient's metabolic state. By combining heterogeneous data types, machine learning models may capture the complex interactions between genetic, environmental, and lifestyle factors that influence metabolic health. Moreover, the predictive machine learning models described herein estimate a patient's future risk of the metabolic state based on their current metabolic states, physical activities, and other personal record of the patients. By identifying high risk food items/activities, the method may provide recommendations to prevent health risk progression and empower patients to take proactive steps to optimize their metabolic health.

### BRIEF DESCRIPTION OF DRAWINGS

FIG. 1 shows a metabolic health manager for monitoring metabolic health of a patient, performing analytics on the metabolic health data, and providing a patient-specific recommendation for treating metabolic health-related concerns, according to one embodiment.

FIG. 2 is a high-level block illustrating an example of a computing device used in either as a client device, application server, and/or database server, according to one embodiment.

FIG. 3 is a block diagram of the system architecture of a patient health management platform, according to one embodiment.

FIG. 4 is a flowchart illustrating a process for training a machine-learning model to output a representation of a patient's metabolic health, according to one embodiment.

FIG. 5 is an illustration of the process for implementing a machine-learning model to predict a patient-specific metabolic response, according to one embodiment.

FIG. 6 is a block diagram of the system architecture of a recommendation module, according to one embodiments.

FIG. 7 is a block diagram of a system architecture for a nutrition insight module, according to one embodiment.

FIG. 8 is a flowchart illustrating a process for generating a recommendation for a patient with classifications of food items generated by a metabolic food model, according to one embodiment.

FIG. 9 is a flowchart illustrating a process for training a metabolic food model, according to one embodiment.

FIG. 10 is a graphic illustrating changes in a user's responses to a food item over time, according to one embodiment.

FIG. 11 is a graphic illustrating changes in classifications of food items determined for a user over time, according to one embodiment.

FIG. 12 is a block diagram of a system architecture for an activity insight, according to one embodiment.

FIG. 13 is a flowchart illustrating a process for generating a notification for a patient with activities recommended by a metabolic activity model, according to one embodiment.

FIG. 14 is a flowchart illustrating a process for training a metabolic activity model, according to one embodiment.

FIG. 15 is an illustration of a notification recommending a patient perform activities in the afternoon, according to one embodiment.

The figures depict various embodiments of the presented invention for purposes of illustration only. One skilled in the art will readily recognize from the following discussion that alternative embodiments of the structures and methods illustrated herein may be employed without departing from the principles described herein.

### DETAILED DESCRIPTION

### I. System Environment

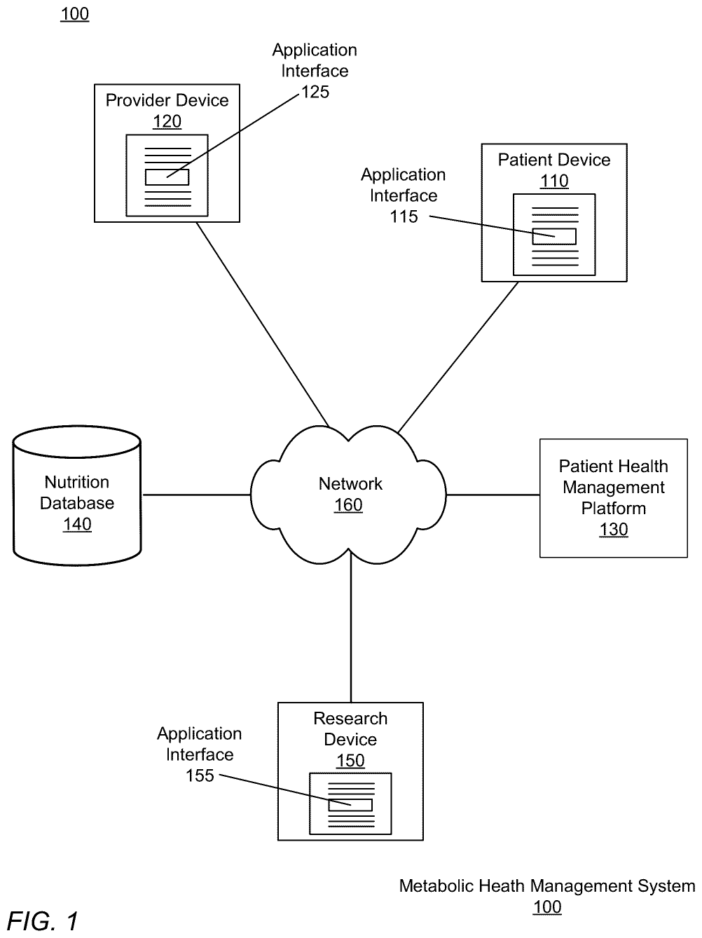

FIG. 1 shows a metabolic health manager 100 for monitoring a patient's metabolic health, for performing analytics on metabolic health data recorded for the patient, and for generating a patient-specific recommendation for treating any metabolic health-related concerns, according to one embodiment. The metabolic health manager 100 includes patient device(s) 110, provider device(s) 120, a patient health management platform 130, a nutrition database 140, research device(s) 150 and a network 160. However, in other embodiments, the system 100 may include different and/or additional components. For example, the patient device 110 can represent thousands or millions of devices for patients (e.g., patient mobile devices) that interact with the system in locations around the world. Similarly, the provider device 120 can represent thousands or millions of devices of providers (e.g., mobile phones, laptop computers, in-provider-office recording devices, etc.). In some cases, a single provider may have more than one device that interacts with the platform 130.

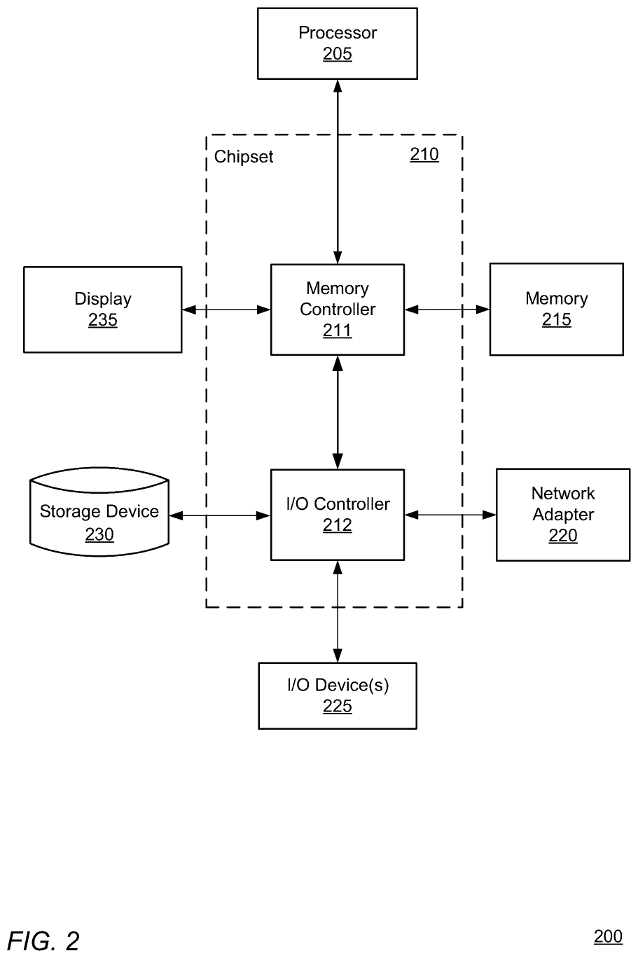

The patient device 110 is a computing device with data processing and data communication capabilities that is capable of receiving inputs from a patient. An example physical implementation is described more completely below with respect to FIG. 2. In addition to data processing, the patient device 110 may include functionality that allows the device 110 to record speech responses articulated by a patient operating the device (e.g., a microphone), and to graphically present data to a patient (e.g., a graphics display). Examples of the patient device 110 include desktop computers, laptop computers, portable computers, GOOGLE HOME, AMAZON ECHO, etc. The patient device 110 may present information generated by the communication platform 130 via a mobile application configured to display and record patient responses. For example, through a software application interface 115, a patient may receive a recommendation or an update regarding their metabolic health.

Application 115 provides a user interface (herein referred to as a “patient dashboard”) that is displayed on a screen of the patient device 110 and allows a patient to input commands to control the operation of the application 115. The patient dashboard enables patients to track and manage changes in a patient's metabolic health. For example, the dashboard allows patients to observe changes in their metabolic health over time, receive recommendation notifications, exchange messages about treatment with a health care provider, and so on. The application 115 may be coded as a web page, series of web pages, or content otherwise coded to render within an internet browser. The application 115 may also be coded as a proprietary application configured to operate on the native operating system of the patient device 110. In addition to providing the dashboard, application 115 may also perform some data processing on biological and food data locally using the resources of patient device 110 before sending the processed data through the network 150. Patient data sent through the network 150 is received by the patient health management platform 130 where it is analyzed and processed for storage and retrieval in conjunction with a database.

Similarly, a provider device 120 is a computing device with data processing and data communication capabilities that is capable of receiving input from a provider. The provider device 120 is configured to present a patient's medical history or medically relevant data (i.e., a display screen). The above description of the functionality of the patient device 110 also can apply to the provider device 120. The provider device 120 can be a personal device (e.g., phone, tablet) of the provider, a medical institution computer (e.g., a desktop computer of a hospital or medical facility), etc. In addition, the provider device 120 can include a device that sits within the provider office such that the patient can interact with the device inside the office. In such implementations, the provider device is a customized device with audio and/or video capabilities (e.g., a microphone for recording, a display screen for text and/or video, an interactive user interface, a network interface, etc.). The provider device 120 may also present information to medical providers or healthcare organizations via a mobile application similar to the application described with reference to patient device 110.

Application 125 provides a user interface (herein referred to as a “provider dashboard”) that is displayed on a screen of the provider device 120 and allows a medical provider or trained professional/coach to input commands to control the operation of the application 125. The provider dashboard enables providers to track and manage changes in a patient's metabolic health. The application 125 may be coded as a web page, series of web pages, or content otherwise coded to render within an internet browser. The application 125 may also be coded as a proprietary application configured to operate on the native operating system of the patient device 110.

The patient health management platform 130 is a medium for dynamically generating recommendations for improving a patient's metabolic health based on biological data recorded from a plurality of sources including wearable sensors (or other types of IoT sensors), lab tests, etc., and food or diet-related data recorded by the patient. The patient health management platform 130 predicts a patient's metabolic response based on periodically recorded patient data (e.g., nutrition data, symptom data, lifestyle data). Accordingly, a patient's metabolic response describes a change in metabolic health for a patient resulting from the food they most recently consumed and their current metabolic health. Based on such a change, the platform 130 generates a recommendation including instructions for a patient to improve their metabolic health or to maintain their improved metabolic health. Additionally, in real-time or near real-time, the patient health management platform 130 may provide feedback to a patient identifying potential inconsistencies or errors in the food or biological data entered manually by the patient based on a comparison of the patient's true metabolic state and their predicted metabolic state.

The nutrition database 140 stores nutrition data extracted from a collection of nutrient sources, for example food or vitamins. Data within the nutrition database 140 may be populated using data recorded by a combination of public sources and third-party entities such as the USDA, research programs, or affiliated restaurants. The stored data may include, but is not limited to, nutrition information (for example, calories, macromolecule measurements, vitamin concentrations, cholesterol measurements, or other facts) for individual foods or types of foods and relationships between foods and metabolic responses (for example, an impact of a given food on insulin sensitivity). Data stored in the nutrition database 140 may be applicable to an entire population (i.e., general nutrition information) or personalized to an individual patient (i.e., a personalized layer of the nutrition database). For example, the nutrition database 140 may store information describing a patient's particular biological (i.e., metabolic) response to a food. In such embodiments, the nutrition database 140 may be updated based on feedback from the patient health management platform 140.

Application 155 provides a user interface (herein referred to as a “research dashboard”) that is displayed on a screen of the research device 150 and allows a researcher to input commands to control the operation of the application 155. The research dashboard enables providers to track and manage changes in a patient's metabolic health. The application 155 may be coded as a web page, series of web pages, or content otherwise coded to render within an internet browser. The application 155 may also be coded as a proprietary application configured to operate on the native operating system of the patient device 110.

Interactions between the patient device 110, the provider device 120, the patient health management platform 130, and the nutrition database 140 are typically performed via the network 150, which enables communication between the patient device 120, the provider device 130, and the patient communication platform 130. In one embodiment, the network 150 uses standard communication technologies and/or protocols including, but not limited to, links using technologies such as Ethernet, 802.11, worldwide interoperability for microwave access (WiMAX), 3G, 4G, LTE, digital subscriber line (DSL), asynchronous transfer mode (ATM), InfiniBand, and PCI Express Advanced Switching. The network 150 may also utilize dedicated, custom, or private communication links. The network 150 may comprise any combination of local area and/or wide area networks, using both wired and wireless communication systems.

FIG. 2 is a high-level block diagram illustrating physical components of an example computer 200 that may be used as part of a client device (e.g., devices 110, 120, 150), application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information for the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

### II. Overview of Metabolic Health Manager

In the United States, treating non-communicable diseases including, but not limited to, diabetes, hyper-tension, high-cholesterol, heart disease, obesity, fatty liver disease, arthritis, irritable bowel syndrome (IBS), and infertility, is a multi-billion-dollar industry. Still, these diseases account for over 2 million deaths annually. Conventional treatments are directed towards addressing and alleviating symptoms of each disease, but they fail to recognize that the root of all the aforementioned diseases is an impaired metabolism. By addressing root cause metabolic impairments, a patient's disease may not just be managed on a per symptom basis, but it may be reversed entirely. Accordingly, a treatment or system for generating a treatment directed towards treating metabolic impairments in patients suffering from such diseases could be more effective and most cost-efficient. Because the patient health management platform 100 aims to treat a patient's metabolic impairments, a patient using the patient health management platform 100 for an extended time period may transition from a first state of impaired metabolism to a second state of functional metabolism to a third state of optimal metabolism.

The patient health management platform 130, as described herein, recognizes that a patient's body is a unique system in a unique state in which metabolism is a core biochemical process. Accordingly, the treatment and nutrition recommendations generated by the platform 130 are tailored to suit a patient's unique metabolic state and the unique parameters or conditions that impact or have previously impacted their metabolic state. To enable a patient to achieve good or optimal metabolic health, the platform 130 records measurements of various factors and aims to improve these measurements to levels representative of an optimized metabolic state. For example, five factors commonly considered include blood sugar, triglycerides, good cholesterol (high-density lipoprotein), blood pressure, and waist circumference. Each human body is different and continuously evolving. To guide a patient towards optimal metabolic health, the platform establishes a deep understanding of the dynamic states of each human body over time by capturing continuous biosignals and deriving insights from these biosignals.

For each patient, the platform 130 leverages a combination of personalized treatments that are tailored to a patient's unique metabolic state based on a combination of timely, accurate, and complete recordings of metabolic biosignals. Such measurements are collectively referred to herein as “TAC measurements.” The platform determines a current metabolic state of a human body by analyzing a unique combination of continuous biosignals received from various sources including, but not limited to, near-real-time data from wearable sensors (e.g. continuous blood glucose, heart rate, etc.), periodic lab tests (e.g., blood work), nutrition data (e.g., macronutrients, micronutrients, and biota nutrients from food and supplements of the patient), medicine data (e.g., precise dosage and time of medications taken by the patient), and symptom data (e.g., headache, cramps, frequent urination, mood, energy, etc., reported by each patient via a mobile app). This analysis is performed continuously to establish a time series of metabolic states. As a result, the platform understands not only the current state of each patient, but also the full history of states that led to the current state. Using a patient's current metabolic state and their full history of metabolic states, the platform is able to deeply personalize the treatment for each patient.

The platform applies various technologies and processing techniques to gain a deep understanding of the combination of factors contributing to a patient's metabolic state and to establish a personalized metabolic profile for each patient. For example, the platform implements a combination of analytics (e.g., analyzing trends, outliers, and anomalies in biosignals as well as correlations across multiple biosignals), rule based artificial intelligence (AI), machine learning-based AI, and automated cohorting or clustering.

For the sake of explanation, the concepts and techniques described herein are described with reference to diabetes. However, one of skill in the art would recognize that the concepts and techniques may also be applied to any other disease resulting from an impaired metabolism. As will be described herein, a patient's metabolic health describes the overall effectiveness of their metabolism. For example, a patient's metabolic health may be categorized as impaired, functional, or optimal. To gain insight into a patient's metabolic health, the patient health management platform 130 identifies metabolic states occurring over a time period and changes between those metabolic states. As described herein, a metabolic state represents a patient's state of metabolic health at a specific time (e.g., a state of metabolic health resulting from consumption of a particular food or adherence to a particular medication/treatment).

In addition, the term “continuously” is used throughout the description to characterize the collection of biosignals and other data regarding the patient. This term can refer to a rate of collection that is truly continuous (e.g., a constantly recorded value) or near continuous (e.g., collection at every time point or time increment, such as every millisecond, second, or minute), such as biosignals recorded by a wearable device. In some cases, continuously recorded data may refer to particular biosignals that occur semi-regularly, such as a lab test that is taken at a recurring time interval (e.g., every 10 minutes, 30 minutes, hour, 5 hours, day or number of days, week or number of weeks, etc.). The term “continuously” does not exclude situations in which wearable sensors may be removed during certain activities or at times of day (e.g., while showering). In other embodiments, the platform collects multiple biosignals that, in combination, represent a continuous or near continuous signal collection even though some biosignals are collected more frequently than others.

### III. Biosignal Data

A patient health management platform receives biosignal data for a patient from a variety of sources including, but not limited to, wearable sensor data, lab test data, nutrition data, medication data, symptom data.

A patient using the metabolic health manager is outfitted with one or more wearable sensors configured to continuously record biosignals, herein referred to as wearable sensor data. Wearable sensor data includes, but is not limited to, biosignals describing a patient's heart rate, record of exercise (e.g., steps, average number of active minutes), quality of sleep (e.g., sleep duration, sleep stages), a blood glucose measurement, a ketone measurement, systolic and diastolic blood pressure measurements, weight, BMI, percentage of fat, percentage of muscle, bone mass measurement, and percent composition of water. A wearable sensor may be a sensor that is periodically removable by a patient (e.g., a piece of jewelry worn in contact with a patient's skin to record such biosignals) or a non-removable device/sensor embedded into a patient's skin (e.g., a glucose patch). Whenever worn or activated to record wearable sensor data, the sensor continuously records one or more of the measurements listed above. In some implementations, a wearable sensor may record different types of wearable sensor data at different rates or intervals. For example, the wearable sensor may record blood glucose measurements, heart rate measurements, and steps in 15 second intervals, but record blood pressure measurements, weight measurements, and sleep trends in daily intervals.

The patient health management platform also receives lab test data recorded for a patient. As described herein, lab test data describes the results of lab tests performed on the patient. Examples of lab test data include, but are not limited to, blood tests or blood draw analysis. Compared to the frequencies at which wearable sensor data is recorded, lab test data may be recorded at longer intervals, for example bi-weekly or monthly. In some implementations, the patient health management platform receives data measured from 126-variable blood tests.

The patient health management platform may also receive nutrition data 320 describing food that a patient is consuming or has consumed. Via an interface (e.g., the application interface) presented on the patient device, a patient enters a record of food that they have consumed on a per meal basis and a time at which each item of food was consumed. Alternatively, the patient may enter the record for food on a daily basis. The patient health management platform extracts nutrition details (e.g., macronutrient, micronutrient, and biota nutrient data) from a nutrition database (not shown) based on the food record entered by the patient. As an example, via a patient device, a patient may record that they consumed two bananas for breakfast at 7:30 AM. The record of the two bananas is communicated to the patient health management platform 350 and the patient health management platform accesses, from a nutrition database, nutrient data including the amount of potassium in a single banana. The accessed nutrient data is returned to the patient health management platform as an update to the recorded nutrition data. Via the same interface or one similar to the interface used to record food consumed, a patient may record and communicate medication data and symptom data to the patient health management platform. Medication data describes a type of medication taken, a time at which the medication was taken, and an amount of the medication taken. In addition to nutrition data and medication data, the patient health management platform may receive descriptions of a patient's energy, mood, or general level of satisfaction with their lifestyle, treatment plan, and disease management.

Examples of biosignal data recorded and communicated to the patient health management platform include, but are not limited to, those listed in Table 1. Table 1 also lists a source for recording each example of biosignal data.

TABLE 1
Example Biosignal Data and Source

| Category | Type | Signal | Source |
|---|---|---|---|
| Sensor Data | Biomarker | Weight | Body Composition Scale |
| | Biomarker | Body fat % | Body Composition Scale |
| | Biomarker | Subcutaneous fat % | Body Composition Scale |
| | Biomarker | Visceral fat % | Body Composition Scale |
| | Biomarker | Body water % | Body Composition Scale |
| | Biomarker | Muscle % | Body Composition Scale |
| | Biomarker | Bone mass | Body Composition Scale |
| | Biomarker | Basal metabolic rate | Body Composition Scale |
| | Biomarker | Protein | Body Composition Scale |
| | Biomarker | Lean body weight | Body Composition Scale |
| | Biomarker | Muscle mass | Body Composition Scale |
| | Biomarker | Metabolic age | Body Composition Scale |
| | Biomarker | Continuous Blood | Continuous Glucose Meter |
| | | Glucose | |
| | Biomarker | Ketones | Ketone Meter |
| | Biomarker | Systolic BP | Blood Pressure Meter |
| | Biomarker | Diastolic BP | Blood Pressure Meter |
| | Heart | Resting Heart Rate | Fitness Watch |
| | Heart | Continuous Heart | Fitness Watch |
| | | Rate | |
| Lab Test Data | Biomarker | Skin Temperature | Patient Investigation/Test |
| | Biomarker | Oxygen Saturation | Patient Investigation/Test |
| | Biomarker | Waist Circumference | Patient Investigation/Test |
| | Biomarker | Age | Patient Interview |
| | Biomarker | Gender | Patient Interview |
| | Biomarker | Height | Patient Interview |
| | Biomarker | BMI | Patient Interview |
| | Biomarker | HbA1c | Blood Test |
| | Biomarker | 5dg-cgm | Blood Test |
| | Biomarker | 1dg-cgm | Blood Test |
| | Biomarker | Insulin | Blood Test |
| | Biomarker | Fructosamine | Blood Test |
| | Biomarker | C-Peptide | Blood Test |
| | Biomarker | HOMA-IR | Blood Test |
| | Biomarker | 5dk | Blood Test |
| | Biomarker | Cholesterol | Blood Test |
| | Biomarker | Triglycerides | Blood Test |
| | Biomarker | HDL Cholesterol | Blood Test |
| | Biomarker | LDL Cholesterol | Blood Test |
| | Biomarker | VLDL Cholesterol | Blood Test |
| | Biomarker | Triglyceride/HDL | Blood Test |
| | | Ratio | |
| | Biomarker | Total Cholesterol/ | Blood Test |
| | | HDL Ratio | |
| | Biomarker | Non - HDL | Blood Test |
| | | Cholesterol | |
| | Biomarker | LDL/HDL Ratio | Blood Test |
| | Biomarker | Total Iron Binding | Blood Test |
| | | Capacity (TIBC) | |
| | Biomarker | Serum Iron | Blood Test |
| | Biomarker | % Transferrin | Blood Test |
| | | Saturation | |
| | Biomarker | Amylase | Blood Test |
| | Biomarker | Lipase | Blood Test |
| | Biomarker | Ferritin | Blood Test |
| | Biomarker | Homocysteine | Blood Test |
| | Biomarker | Magnesium | Blood Test |
| | Biomarker | ALT | Blood Test |
| | Biomarker | AST | Blood Test |
| | Biomarker | ALP | Blood Test |
| | Biomarker | Total Bilirubin | Blood Test |
| | Biomarker | Direct Bilirubin | Blood Test |
| | Biomarker | Indirect Bilirubin | Blood Test |
| | Biomarker | Gamma Glutamyl | Blood Test |
| | | Transferase (GGT) | |
| | Biomarker | Protein | Blood Test |
| | Biomarker | Albumin | Blood Test |
| | Biomarker | A/G Ratio | Blood Test |
| | Biomarker | Globulin | Blood Test |
| | Biomarker | Urea | Blood Test |
| | Biomarker | Creatinine | Blood Test |
| | Biomarker | Uric Acid | Blood Test |
| | Biomarker | GFR | Blood Test |
| | Biomarker | Blood urea nitrogen | Blood Test |
| | | (BUN) | |
| | Biomarker | BUN/Creatinine | Blood Test |
| | | Ratio | |
| | Biomarker | Lipoprotein(a) | Blood Test |
| | Biomarker | Apolipoprotein A1 | Blood Test |
| | Biomarker | ApoB | Blood Test |
| | Biomarker | hs-CRP | Blood Test |
| | Biomarker | Apo B/Apo A1 | Blood Test |
| | | Ratio | |
| | Biomarker | LP-PLA2 | Blood Test |
| | Biomarker | Total Triiodothyronine | Blood Test |
| | | [T3] | |
| | Biomarker | Total Thyroxine [T4] | Blood Test |
| | Biomarker | TSH | Blood Test |
| | Biomarker | Sodium | Blood Test |
| | Biomarker | Chloride | Blood Test |
| | Biomarker | Potassium | Blood Test |
| | Biomarker | Bicarbonate | Blood Test |
| | Biomarker | Calcium | Blood Test |
| | Biomarker | Phosphorous | Blood Test |
| | Biomarker | Anion Gap | Blood Test |
| | Biomarker | Vitamin A | Blood Test |
| | Biomarker | Vitamin D2 | Blood Test |
| | Biomarker | Vitamin D3 | Blood Test |
| | Biomarker | Vitamin D Total | Blood Test |
| | Biomarker | Vitamin E | Blood Test |
| | Biomarker | Vitamin K | Blood Test |
| | Biomarker | Vitamin B1/Thiamin | Blood Test |
| | Biomarker | Vitamin B2/ | Blood Test |
| | | Riboflavin | |
| | Biomarker | Vitamin B3/ | Blood Test |
| | | Nicotinic Acid | |
| | Biomarker | Vitamin B5/ | Blood Test |
| | | Pantothenic Acid | |
| | Biomarker | Vitamin B6/ | Blood Test |
| | | Pyridoxal-5- | |
| | | Phosphate | |
| | Biomarker | Vitamin B7/Biotin | Blood Test |
| | Biomarker | Vitamin B9/Folic | Blood Test |
| | | Acid | |
| | Biomarker | Vitamin B12/ | Blood Test |
| | | Cobalamin | |
| | Biomarker | Cortisol | Blood Test |
| | Biomarker | Cystatin C | Blood Test |
| | Biomarker | Serum Zinc | Blood Test |
| | Biomarker | Serum Copper | Blood Test |
| | Biomarker | Basophils - | Blood Test |
| | | Absolute Count | |
| | Biomarker | Eosinophils - | Blood Test |
| | | Absolute Count | |
| | Biomarker | Lymphocytes - | Blood Test |
| | | Absolute Count | |
| | Biomarker | Monocytes - | Blood Test |
| | | Absolute Count | |
| | Biomarker | Mixed - | Blood Test |
| | | Absolute Count | |
| | Biomarker | Neutrophils - | Blood Test |
| | | Absolute Count | |
| | Biomarker | Basophils | Blood Test |
| | Biomarker | Eosinophils | Blood Test |
| | Biomarker | Immature | Blood Test |
| | | Granulocytes (Ig) | |
| | Biomarker | Immature | Blood Test |
| | | Granulocyte | |
| | | Percentage (Ig %) | |
| | Biomarker | White Blood Cells | Blood Test |
| | | (Leucocytes Count) | |
| | Biomarker | Lymphocyte | Blood Test |
| | | Percentage | |
| | Biomarker | Mean Corpuscular | Blood Test |
| | | Hemoglobin (Mch) | |
| | Biomarker | Mean Corp. Hemo. | Blood Test |
| | | Conc. (Mchc) | |
| | Biomarker | MCV | Blood Test |
| | Biomarker | Monocytes | Blood Test |
| | Biomarker | Mean Platelet | Blood Test |
| | | Volume (Mpv) | |
| | Biomarker | Neutrophils | Blood Test |
| | Biomarker | Nucleated Red Blood | Blood Test |
| | | Cells | |
| | Biomarker | Nucleated Red Blood | Blood Test |
| | | Cells % | |
| | Biomarker | Plateletcrit (Pct) | Blood Test |
| | Biomarker | Hematocrit | Blood Test |
| | Biomarker | Platelet Distribution | Blood Test |
| | | Width (Pdw- SD) | |
| | Biomarker | Platelet To Large Cell | Blood Test |
| | | Ratio (Plcr) | |
| | Biomarker | Platelet Count | Blood Test |
| | Biomarker | Red Blood Cell Count | Blood Test |
| | Biomarker | Red Cell Distribution | Blood Test |
| | | Width (Rdw-Cv) | |
| | Biomarker | Red Cell Distribution | Blood Test |
| | | Width - Sd (Rdw-Sd) | |
| | Biomarker | Blood pH | Blood Test |
| | Biomarker | Hemoglobin | Blood Test |
| | Biomarker | ACCP | Blood Test |
| | Biomarker | ANA | Blood Test |
| | Biomarker | Cadmium | Blood Test |
| | Biomarker | Cobalt | Blood Test |
| | Biomarker | Chromium | Blood Test |
| | Biomarker | Caesium | Blood Test |
| | Biomarker | Mercury | Blood Test |
| | Biomarker | Manganese | Blood Test |
| | Biomarker | Molybdenum | Blood Test |
| | Biomarker | Nickel | Blood Test |
| | Biomarker | Lead | Blood Test |
| | Biomarker | Antimony | Blood Test |
| | Biomarker | Selenium | Blood Test |
| | Biomarker | Tin | Blood Test |
| | Biomarker | Strontium | Blood Test |
| | Biomarker | Thallium | Blood Test |
| | Biomarker | Uranium | Blood Test |
| | Biomarker | Vanadium | Blood Test |
| | Biomarker | Silver | Blood Test |
| | Biomarker | Aluminium | Blood Test |
| | Biomarker | Arsenic | Blood Test |
| | Biomarker | Barium | Blood Test |
| | Biomarker | Beryllium | Blood Test |
| | Biomarker | Bismuth | Blood Test |
| | Biomarker | Testosterone | Blood Test |
| Lifestyle Data | Sleep | Sleep Quality | Fitness Watch |
| | Sleep | Minutes Asleep | Fitness Watch |
| | Sleep | Minutes Awake | Fitness Watch |
| | Sleep | Minutes Light Sleep | Fitness Watch |
| | Sleep | Minutes Deep Sleep | Fitness Watch |
| | Sleep | Minutes REM Sleep | Fitness Watch |
| | Exercise | Activity Calories | Fitness Watch |
| | Exercise | Marginal Calories | Fitness Watch |
| | Exercise | BMR Calories | Fitness Watch |
| | Exercise | Total Calories Burned | Fitness Watch |
| | Exercise | Continuous Steps (per | Fitness Watch |
| | | minute) | |
| | Exercise | Fairly Active Minutes | Fitness Watch |
| | Exercise | Light Active Minutes | Fitness Watch |
| | Exercise | Very Active Minutes | Fitness Watch |
| | Exercise | Sedentary Minutes | Fitness Watch |
| | Exercise | Stress | Fitness Watch |
| | Patient | Age | Patient Interview |
| | Information | | |
| | Patient | Gender | Patient Interview |
| | Information | | |
| | Patient | Height | Patient Interview |
| | Information | | |
| | Patient | BMI | Patient Interview |
| | Information | | |
| | Patient | Vegetarian | Patient Interview |
| | Information | | |
| | Patient | Tobacco | Patient Interview |
| | Information | | |
| | Patient | Alcohol | Patient Interview |
| | Information | | |
| | Patient | Caffeine | Patient Interview |
| | Information | | |
| | Family | Father Diabetic? | Patient Interview |
| | Information | | |
| | Family | Mother Diabetic? | Patient Interview |
| | Information | | |
| | Family | Sibling Diabetic? | Patient Interview |
| | Information | | |
| | Family | Grandparents Diabetic? | Patient Interview |
| | Information | | |
| | Happiness | Energy | Patient Health Management App |
| | Happiness | Mood | Patient Health Management App |
| | Happiness | Cuisine Preferences | Patient Health Management App |
| | Happiness | Food Ratings | Patient Health Management App |
| | Happiness | Meal Ratings | Patient Health Management App |
| | Happiness | Exercise Preferences | Patient Health Management App |
| Symptom Data | Symptom | Headache | Patient Health Management App |
| | Symptom | Cramps | Patient Health Management App |
| | Symptom | Numbness | Patient Health Management App |
| | Symptom | Frequent Urination | Patient Health Management App |
| | Symptom | Blurred Vision | Patient Health Management App |
| | Symptom | Tiredness | Patient Health Management App |
| | Symptom | Excess hunger | Patient Health Management App |
| | Symptom | Giddiness | Patient Health Management App |
| | Symptom | Nausea | Patient Health Management App |
| | Symptom | Vomiting | Patient Health Management App |
| | Symptom | Diarrhea | Patient Health Management App |
| | Symptom | Excess thirst | Patient Health Management App |
| | Symptom | Constipation | Patient Health Management App |
| | Symptom | Erectile dysfunction | Patient Health Management App |
| | Symptom | Sleeplessness | Patient Health Management App |
| Medication Data | Medication | Diabetes Medicine | Patient Health Management App |
| | Medication | Insulin | Patient Health Management App |
| | Medication | Hypertension | Patient Health Management App |
| | | Medicines | |
| | Medication | Cholesterol | Patient Health Management App |
| | | Medicines | |
| | Medication | Obesity Medicines | Patient Health Management App |
| | Medication | Heart Medicines | Patient Health Management App |
| | Medication | Arthritis Medicines | Patient Health Management App |
| Nutrition Data | Macronutrients | Net Carb | Nutrition Database/Patient |
| | | | Health Management App |
| | Macronutrients | Calories consumed | Nutrition Database/Patient |
| | | | Health Management App |
| | Macronutrients | Net GI Carb | Nutrition Database/Patient |
| | | | Health Management App |
| | Macronutrients | Fiber | Nutrition Database/Patient |
| | | | Health Management App |
| | Macronutrients | Fat | Nutrition Database/Patient |
| | | | Health Management App |
| | Macronutrients | Protein | Nutrition Database/Patient |
| | | | Health Management App |
| | Macronutrients | Total Carb | Nutrition Database/Patient |
| | | | Health Management App |
| | Micronutrients | Fructose | Nutrition Database/Patient |
| | | | Health Management App |
| | Micronutrients | Sodium | Nutrition Database/Patient |
| | | | Health Management App |
| | Micronutrients | Potassium | Nutrition Database/Patient |
| | | | Health Management App |
| | Micronutrients | Magnesium | Nutrition Database/Patient |
| | | | Health Management App |
| | Micronutrients | Calcium | Nutrition Database/Patient |
| | | | Health Management App |
| | Micronutrients | Chromium | Nutrition Database/Patient |
| | | | Health Management App |
| | Micronutrients | Omega 3 | Nutrition Database/Patient |
| | | | Health Management App |
| | Micronutrients | Omega 6 | Nutrition Database/Patient |
| | | | Health Management App |
| | Micronutrients | ALA | Nutrition Database/Patient |
| | | | Health Management App |
| | Micronutrients | Q10 | Nutrition Database/Patient |
| | | | Health Management App |
| | Micronutrients | Biotin | Nutrition Database/Patient |
| | | | Health Management App |
| | Micronutrients | Flavonoids | Nutrition Database/Patient |
| | | | Health Management App |
| | Glycemic | Improve IS | Nutrition Database/Patient |
| | Controllers | | Health Management App |
| | Glycemic | Inhibit GNG | Nutrition Database/Patient |
| | Controllers | | Health Management App |
| | Glycemic | Inhibit Carb | Nutrition Database/Patient |
| | Controllers | Absorption | Health Management App |
| | Glycemic | Improve Insulin | Nutrition Database/Patient |
| | Controllers | Secretion | Health Management App |
| | Glycemic | Impr B-Cell Regen | Nutrition Database/Patient |
| | Controllers | | Health Management App |
| | Glycemic | Inhibit Hunger | Nutrition Database/Patient |
| | Controllers | | Health Management App |
| | Glycemic | Inhibit Glucose | Nutrition Database/Patient |
| | Controllers | Kidney Reabsorption | Health Management App |
| | Biotanutrients | Lactococcus sp. | Nutrition Database/Patient |
| | | | Health Management App |
| | Biotanutrients | Lactobacillus sp. | Nutrition Database/Patient |
| | | | Health Management App |
| | Biotanutrients | Leuconostoc sp. | Nutrition Database/Patient |
| | | | Health Management App |
| | Biotanutrients | Streptococcus sp. | Nutrition Database/Patient |
| | | | Health Management App |
| | Biotanutrients | Bifidobacterium sp. | Nutrition Database/Patient |
| | | | Health Management App |
| | Biotanutrients | Saccharomyces sp. | Nutrition Database/Patient |
| | | | Health Management App |
| | Biotanutrients | Bacillus sp. | Nutrition Database/Patient |
| | | | Health Management App |
| | Glycemic | Glycemic Index | Nutrition Database/Patient |
| | Impact | | Health Management App |
| | Fats | Saturated fat | Nutrition Database/Patient |
| | | | Health Management App |
| | Fats | Monounsaturated fat | Nutrition Database/Patient |
| | | | Health Management App |
| | Fats | Polyunsaturated fat | Nutrition Database/Patient |
| | | | Health Management App |
| | Fats | Trans fat | Nutrition Database/Patient |
| | | | Health Management App |
| | Fats | Cholesterol | Nutrition Database/Patient |
| | | | Health Management App |
| | Proteins | Histidine | Nutrition Database/Patient |
| | | | Health Management App |
| | Proteins | Isoleucine | Nutrition Database/Patient |
| | | | Health Management App |
| | Proteins | Lysine | Nutrition Database/Patient |
| | | | Health Management App |
| | Proteins | Methionine + | Nutrition Database/Patient |
| | | Cysteine | Health Management App |
| | Proteins | Phenylalanine + | Nutrition Database/Patient |
| | | Tyrosine | Health Management App |
| | Proteins | Tryptophan | Nutrition Database/Patient |
| | | | Health Management App |
| | Proteins | Threonine | Nutrition Database/Patient |
| | | | Health Management App |
| | Proteins | Valine | Nutrition Database/Patient |
| | | | Health Management App |
| | Vitamins/Minerals | Vitamin A | Nutrition Database/Patient |
| | | | Health Management App |
| | Vitamins/Minerals | Vitamin C | Nutrition Database/Patient |
| | | | Health Management App |
| | Vitamins/Minerals | Vitamin D | Nutrition Database/Patient |
| | | | Health Management App |
| | Vitamins/Minerals | Vitamin E | Nutrition Database/Patient |
| | | | Health Management App |
| | Vitamins/Minerals | Vitamin K | Nutrition Database/Patient |
| | | | Health Management App |
| | Vitamins/Minerals | B1 | Nutrition Database/Patient |
| | | | Health Management App |
| | Vitamins/Minerals | B12 | Nutrition Database/Patient |
| | | | Health Management App |
| | Vitamins/Minerals | B2 | Nutrition Database/Patient |
| | | | Health Management App |
| | Vitamins/Minerals | B3 | Nutrition Database/Patient |
| | | | Health Management App |
| | Vitamins/Minerals | B5 | Nutrition Database/Patient |
| | | | Health Management App |
| | Vitamins/Minerals | B6 | Nutrition Database/Patient |
| | | | Health Management App |
| | Vitamins/Minerals | Folate | Nutrition Database/Patient |
| | | | Health Management App |
| | Vitamins/Minerals | Copper | Nutrition Database/Patient |
| | | | Health Management App |
| | Vitamins/Minerals | Iron | Nutrition Database/Patient |
| | | | Health Management App |
| | Vitamins/Minerals | Zinc | Nutrition Database/Patient |
| | | | Health Management App |
| | Vitamins/Minerals | Manganese | Nutrition Database/Patient |
| | | | Health Management App |
| | Vitamins/Minerals | Phosphorus | Nutrition Database/Patient |
| | | | Health Management App |
| | Vitamins/Minerals | Selenium | Nutrition Database/Patient |
| | | | Health Management App |
| | Vitamins/Minerals | Omega 6/omega 3 | Nutrition Database/Patient |
| | | | Health Management App |
| | Vitamins/Minerals | Zinc/Copper | Nutrition Database/Patient |
| | | | Health Management App |
| | Vitamins/Minerals | Potassium/Sodium | Nutrition Database/Patient |
| | | | Health Management App |
| | Vitamins/Minerals | Calcium/Magnesium | Nutrition Database/Patient |
| | | | Health Management App |
| | Vitamins/Minerals | PRAL Alkalinity | Nutrition Database/Patient |
| | | | Health Management App |
| | Metabolic | Improve BP | Nutrition Database/Patient |
| | Improvers | | Health Management App |
| | Metabolic | Improve Cholesterol | Nutrition Database/Patient |
| | Improvers | | Health Management App |
| | Metabolic | Reduce Weight | Nutrition Database/Patient |
| | Improvers | | Health Management App |
| | Metabolic | Improve Renal | Nutrition Database/Patient |
| | Improvers | function | Health Management App |
| | Metabolic | Improve Liver | Nutrition Database/Patient |
| | Improvers | function | Health Management App |
| | Metabolic | Improve Thyroid | Nutrition Database/Patient |
| | Improvers | function | Health Management App |
| | Metabolic | Improve Arthritis | Nutrition Database/Patient |
| | Improvers | | Health Management App |
| | Metabolic | Reduce uric acid | Nutrition Database/Patient |
| | Improvers | | Health Management App |
| | Food Type | Fruits | Nutrition Database/Patient |
| | | | Health Management App |
| | Food Type | Oils | Nutrition Database/Patient |
| | | | Health Management App |
| | Food Type | Spices | Nutrition Database/Patient |
| | | | Health Management App |
| | Food Type | Grains | Nutrition Database/Patient |
| | | | Health Management App |
| | Food Type | Legumes | Nutrition Database/Patient |
| | | | Health Management App |
| | Food Type | Nuts | Nutrition Database/Patient |
| | | | Health Management App |
| | Food Type | Seed Products | Nutrition Database/Patient |
| | | | Health Management App |
| | Cellular | Inflammatory index | Nutrition Database/Patient |
| | Stressors | | Health Management App |
| | Cellular | Oxidative stress index | Nutrition Database/Patient |
| | Stressors | | Health Management App |
| | Cellular | Gluten | Nutrition Database/Patient |
| | Stressors | | Health Management App |
| | Cellular | Lactose | Nutrition Database/Patient |
| | Stressors | | Health Management App |
| | Cellular | Alcohol | Nutrition Database/Patient |
| | Stressors | | Health Management App |
| | Cellular | Allergic index | Nutrition Database/Patient |
| | Stressors | | Health Management App |
| | Hydration | Water | Nutrition Database/Patient |
| | | | Health Management App |

### IV. Patient Health Management Platform

### IV.A General System Architecture

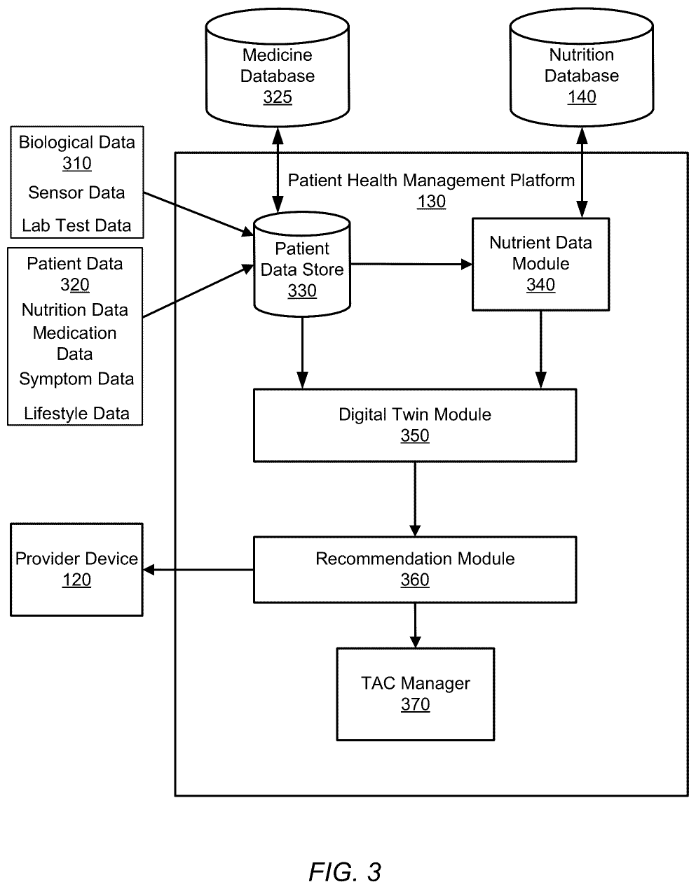

FIG. 3 is a block diagram of the system architecture of the patient health management platform 130, according to one embodiment. The patient health management platform 130 includes a patient data store 330, a nutrient data module 340, a digital twin module 350, a recommendation module 360, and a TAC manager 370. However, in other embodiments, the patient health management platform 130 may include different and/or additional components.

The patient health management platform 130 receives biological data 310 recorded by a variety of technical sources. Biological data 310 includes sensor data comprising biosignals recorded by one or more sensors worn or implemented by a patient. Such biosignals are continuously recorded and each recorded biosignal is assigned a timestamp indicating when it was recorded. Biological data 310 further includes lab test data determined based on blood draw analysis and/or other examinations that a patient has been subjected. Biosignals collected through lab test data may be recorded less frequently than biosignals collected through sensor data, for example over bi-weekly or monthly intervals. In some implementations, lab test data is determined based on procedures and analysis performed manually be doctors or researchers or based on analysis performed by machines and computers separate from the metabolic health manager 1000. The patient data store 330 stores biological data 310.

The patient health management platform 130 also receives patient data 320 that is recorded manually by a patient via an application interface on a patient device 110. Patient data 320 includes nutrition data, medication data, symptom data, and lifestyle data. Nutrition data describes a record of foods that a patient has consumed. In some implementations, nutrition data also includes a timestamp indicating when each food was consumed by the patient and a quantity in which each food was consumed. Similarly, medication data describes a record of medications that a patient has taken and, optionally, a timestamp indicating when a patient took each medication and a quantity in which each medication was taken. In response to a patient recording medication data, the patient health management platform may access additional information from a medication database (not shown) to supplement the medication data recorded by the patient. Symptom data describes a record of symptoms experienced by a patient and a timestamp indicating when each symptom was experienced. Lifestyle data describes a record of a patient's physical activity (e.g., exercise) and a record of a patient's sleep history. Lifestyle data may also include a description or selection of emotions or feelings capturing the patient's current state of mind and body (i.e., tired, sore, energetic). In one implementation, each type of patient data 320 may be recorded instantaneously throughout the day when the patient consumes a food, takes a medication, experiences a symptom, or experiences a change in an aspect of their lifestyle. In an alternate implementation, at the end of a day, the patient health management platform 130 detects that a patient has not instantaneously recorded patient data throughout the day and prompts the patient to input a complete record of patient data for the entire day at that time. In addition to biological data 310, the patient data store 330 stores patient data 320.

In some embodiments, the patient data store 330 stores biological data 310 and patient data 330 as an ongoing recorded timeline of entries for a current time period. As new patient data or biological data is recorded or as updates to existing patient data and biological data are received, the patient data store 330 updates the timeline of entries to reflect the new or updated data. Accordingly, the timeline of entries stored in the patient data store 330 comprises foods consumed by the patient at recorded times over the current time period, medications taken by the patient at recorded times over the current time period, and symptoms experienced by the patient at recorded times over the current time period. Some patient data entries may be recorded and reflected in the timeline on a daily basis, whereas other entries are recorded by a patient multiple times a day. Entries for biological data 310, for example, lab test data may be recorded even less frequently, for example as weekly updates to the ongoing timeline. The range of time between a start time and an end time for the current time period may be adjusted manually or trained over time based on predicted and true metabolic states for a patient.

The nutrient data module 340 receives nutrition data from the patient data store 330 and communicates the nutrition data to the nutrition database 140. As described above with reference to FIG. 1, the nutrition database 140 includes comprehensive nutrition information comprising macronutrient information (e.g., protein, fat, carbohydrates), micronutrient information (e.g., Vitamin A, Vitamin B, Vitamin C, sodium, magnesium), and biota nutrients (e.g. lactococcus, lactobacillus) for a wide variety of foods and ingredients. In some implementations, the nutrient data module 340 stores nutrition information in a lookup table or combination of lookup tables organized by food item or a category of food item. In other implementations, the nutrient data module 340 stores nutrition information in a lookup table organized by nutrient information or another suitable system. Based on the nutrition data received from the patient data store 330, the nutrient data module 340 identifies nutrition information associated with each food item of the nutrition data and supplements the nutrition data in the patient data store 330 with the identified nutrition information from the nutrition database 140. In some implementations, the nutrient database 140 includes over 100 food-related attributes including, but not limited to, different types of fat, protein, vitamins, and minerals.

The digital twin module 350 generates a digital replica of the patient's metabolic health based on a combination of biological data 310 and patient data 320, hereafter referred to as a digital twin. The digital twin module 350 considers different aspects of a patient's health and well-being to generate and continuously update a patient's digital twin. As described herein, a digital twin is a dynamic digital representation of the metabolic function of a patient's human body. The digital twin module 350 continuously monitors biological data and patient data and correlates a patient's metabolic history with their ongoing medical history to identify changes in the patient's metabolic state. In one embodiment, the digital twin module implements two sets of trained machine-learning metabolic models: a first set of models trained to predict the patient's metabolic state given patient data as inputs and a second set of models trained to determine the patient's true metabolic state given biological data as inputs.

Based on nutrition data, medication data, symptom data, lifestyle data, and supplemental nutrition information retrieved by the nutrient data module 340, the digital twin module 350 generates a prediction of the patient's metabolic state (herein referred to as a patient's “predicted metabolic state”). The digital twin module 350 implements one or more machine-learning, metabolic models to analyze the patient data 320 recorded over a given time period to generate a prediction of the patient's metabolic state for that time period. Accordingly, the prediction of the patient's metabolic state is a function of a large number of metabolic factors recorded in the patient data 320 (e.g., fasting blood glucose, sleep, and exercise) and a nutrition profile (e.g., macronutrients, micronutrients, biota nutrients).

In addition to the predicted metabolic state, the digital twin module 350 may implement one or more metabolic models to generate a true representation of a patient's metabolic state (herein referred to as a “true metabolic state”) based on the biological data 310 recorded for a time period. In comparison to the metabolic models used to generate a prediction of a patient's metabolic model, the metabolic models implemented by the digital twin module 4350 to determine the true metabolic state of the patient are trained to process aspects of biological data 310 (e.g., wearable sensor data and lab test data) into an affect the patient's metabolic state. For such implementations, at the conclusion of a time period, a metabolic model may be trained to analyze biological data 310 recorded by wearable sensors during the time period and determined based on lab tests from the time period to determine a true metabolic state for the patient that reflects the actual biological conditions experienced by a patient (e.g., their HbA1c levels, or BMI) during the time period. Accordingly, given biological data 310 as an input, the metabolic model is further trained to output a patient's actual biological response (e.g., a measured insulin sensitivity or change in glucose in response to consuming a food or taking a medication).

In some embodiments, digital twin module 350 communicates both the predicted metabolic state and the true metabolic state to the timeliness, accuracy, and completeness (TAC) manager 370. The TAC manager 370 compares the predicted metabolic state and the true metabolic state to determine whether the two states are within a threshold level of similarity to each other. If the two states are within a threshold level of similarity, the TAC manager 370 confirms the timeliness, accuracy, and completeness of the recorded patient data. As described herein, accurately recorded nutrition data, medication data, symptom data, and lifestyle data is accurate in what was recorded in the entry and when the entry was recorded. Alternatively, if the two states are not within a threshold level of similarity, the TAC manager 370 detects that there is an error in the record of the patient data 320. Examples of such errors detected by the TAC manager 370 include, but are not limited to, an entry recorded in an incorrect amount, a failure to record an entry, or an entry recorded at the wrong time. Based on the inconsistency, or inconsistencies, between the true metabolic state and the predicted metabolic state, the TAC manager 370 identifies one or more potential errors in the recorded patient data which may have contributed to the one or more inconsistencies and generates notifications to the patient device 110 for presentation to the patient.

Patient data and biological data may be recorded at varying intervals. For example, sensor data is recorded continuously every 15 minutes, lab test data is recorded bi-weekly, and patient data 320 is recorded multiple times a day as needed. Therefore, the patient health management platform 130 may not receive an updated recording for every type of data in time to generate a predicted metabolic state. When generating a predicted metabolic state for a particular time period, the digital twin module 350 retrieves all patient data 320 recorded within that time period and the metabolic state predicted by the during the preceding time period. In some embodiments, the digital twin module 350 implements one or more machine learning models to process, as inputs, the recorded patient data and the most recently predicted metabolic state into a predicted metabolic state for a current time period. In place of the most recent predicted metabolic state, the digital twin module 350 may input the most recent true metabolic state to the one or more machine learning models. Accordingly, the predicted metabolic state reflects any effects that the most recently recorded patient data 320 had on a previous metabolic state.

Similarly, when generating a true metabolic state for a time period, the digital twin module 350 retrieves all biological data 310 recorded within that time period (e.g., heart rate, exercise, continuous blood glucose, ketones, blood pressure, weight) and the true metabolic state generated during the preceding time period. The digital twin module 350 may also rely on one or more machine learning models to process the retrieved biological data 310 and the most recent true metabolic state into a current true metabolic state. Accordingly, the generated true metabolic state also reflects any effects of the most recently recorded biological data 310 had on a previous metabolic state. For example, a machine learned model may use a continuous blood glucose signal measured every 15 minutes to calculate a patient's 5-day average blood glucose. The computed measurement is compared against established ranges in the medical literature to determine whether the patients are in a diabetic, pre-diabetic, or non-diabetic state as they progress with their treatment. In common implementations, the digital twin module 350 updates a patient's metabolic state at a higher frequency than a frequency at which lab test data is recorded. As such, when lab test data is unavailable for the current time period, the digital twin module 350 may generate the updated metabolic state based on the lab test data recorded most recently for a preceding time period.

In one embodiment, the recommendation module 360 compares a patient's predicted metabolic state to baseline metabolic state for a patient with a functional metabolism. For patients who already have a functional metabolism, the recommendation module 360 compares the predicted metabolic state to a baseline metabolic state for a patient with an optimal metabolism. In either implementation, the recommendation module 360 determines discrepancies between the patient-specific predicted metabolic state and the baseline metabolic state and identifies one or more biosignals which could be adjusted such that the predicted state becomes more similar to the baseline state, for example lower blood glucose levels in the predicted metabolic state or an imbalance between certain micronutrients and micronutrients.

Based on the determined adjustments, the recommendation module 360 generates a recommendation for improving the patient's biosignals to more closely resemble those of the baseline metabolic state. The recommendation includes a set of objectives for a patient to complete to improve the patient's metabolic health. The set of objectives include a medication regimen or schedule, a food or meal schedule, micronutrient and biota nutrient supplements, one or more lifestyle adjustments, or a combination thereof. The medication regimen, food schedule, and supplement schedule may prescribe medications, food items, or supplements which may either replenish nutrients in which a patient is deficient, offset the effects of nutrients for which a patient has an excess, or a combination thereof. The medication regimen, food schedule, and supplement schedule may also alleviate or mitigate the symptoms (as indicated by symptom data recorded by a patient) that a patient is experiencing by addressing the biological root cause of the symptoms.

One example of a medication regimen may include a recommended medication or combination of medications and an adherence schedule for each medication. One example of a food schedule may include a recommended food item or, more broadly, a category of food item and an amount of the food item to be consumed. Similarly, a lifestyle adjustment may prescribe particular lifestyle adjustments for addressing a patient's symptoms or nutrient abnormalities. Examples of lifestyle adjustments include, but are not limited to, increasing physical activity or increasing a patient's amount of sleep. In some implementations, the content of lifestyle adjustments may broadly overlap with food or medication adjustments. For example, a lifestyle adjustment may recommend a patient replace refined carbohydrates with wholegrain foods, while the food schedule includes a set of particular wholegrain foods.

The recommendation module 360 may include a combination of rule-based artificial intelligence techniques representing codified medical knowledge from established medical practice (e.g., American Diabetes Association guidelines, research literature, and insights gained from past medical treatments). The recommendation module 360 applies the codified knowledge in an automated manner to recommend treatments for new patients using the patient health management platform 130.

The recommendation module 360 classifies food items previously recorded by a patient based on the effect of an individual food item on the patient's metabolic state. For example, when a patient consumes a food item, their metabolic state may improve, deteriorate, or remain unchanged. The recommendation module 360 implements a trained machine-learning model to predict an effect of a food item on the metabolic state of the patient and classifies the food item based on the predicted effect. Over time, a patient's metabolic state may change (e.g., improve or worsen) causing the effect of some food items on their metabolic state to also change. For example, the recommendation module 360 may initially classify apples as detrimental to a patient's metabolic state. As the patient's metabolic state improves, apples may no longer be detrimental to the patient's metabolic state. To account for such changes in a patient's metabolic state, the recommendation module 360 classifies food items based on a patient's current metabolic health and compares the current classification with a classification determined for a previous time period. If the classification has changed, the recommendation module 360 generates a notification for the patient indicating that updated classification of the food item (e.g., whether the patient should start consuming the food item or stop consuming the food item). Additionally, because each patient's metabolic state is unique, the recommendation module 360 may implement a patient-specific metabolic model to predict the effect of a food item on the patient's current metabolic state.

The recommendation module 360 also identifies activities (e.g., walking, swimming, bicycling, etc.) to be performed by the patient based on the effect of each activity on the patient's metabolic state. For example, when a patient performs a physical activity, their metabolic state may improve, deteriorate, or remain unchanged. The recommendation module 360 implements a trained machine-learning model to predict an effect of the activity on the metabolic state of the patient and classifies the activity based on the predicted effect. The recommendation module 360 identifies activities that improve the metabolic state of the patient and generates a notification for the patient with a recommendation that the patient perform the identified activities. Additionally, because each patient's metabolic state is unique, the recommendation module 360 may implement a patient-specific metabolic model to predict the effect of an activity on the patient's current metabolic state.

In some implementations, the recommendation module 360 further recommends when a patient should perform the identified activities. For example, the effect of performing an activity at night may be less beneficial to a patient than if the patient performed the activity in the morning. In such implementations, the recommendation module 360 implements another machine-learning model to predict the effect of an activity when performed at a given time during the day. The recommendation module 360 identifies the times predicted to have the most beneficial effects on the patient's metabolic state and updates the recommendation with suggested times for the patient to perform each identified activity.

Additionally, over time, a patient's metabolic state may change (e.g., improve or worsen) causing the effect of some activities to also change. For example, the recommendation module 360 may initially classify walking as having no effect on a patient's metabolic state. As the patient's metabolic state improves, walking may have the effect of improving their metabolic health. To account for such changes in a patient's metabolic state, the recommendation module 360 predicts the effect of the activity on the current metabolic state of the patient and compares the predicted effect with a prediction for a previous time period.

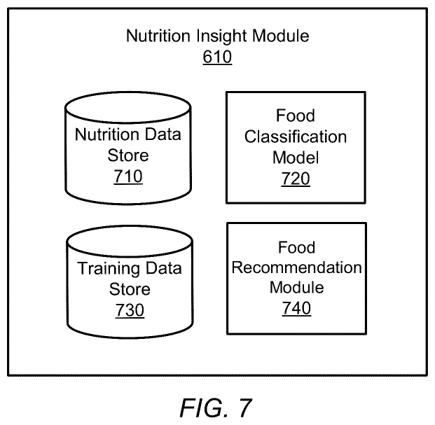

The recommendation module 360 is further described below with reference to FIGS. 7-15.

### IV.B Machine-Learning Metabolic Models

Because the human body is a complex system and different patients may respond differently to the same input stimuli, the patient health management system 130 includes mathematical models trained to learn the relationships between response signals representing a patient's metabolic states and input stimuli causing those responses. As described above, the patient health management system 130 applies machine-learning based artificial intelligence to generate a precision treatment recommendation for improving a patient's metabolic health by predicting their response to future input stimuli. The digital twin module 350 implements a combination of machine-learning models that are iteratively trained to predict a response of the human body based on each patient's current metabolic state and a set of inputs (e.g., recorded patient data, sensor data, and biological data). Each machine-learning model enables the digital twin module 350 to automatically analyze a large combination of biosignals recorded for each patient to characterize a patient's current or potential metabolic state.

In order to model a patient's metabolic state and to track changes in their metabolic health, a model, such as a mathematical function or other more complex logical structure, is trained using the combination of input biosignals described above, to determine a set of parameter values that are stored in advance and used as part of the metabolic analysis. Briefly, a representation of a patient's metabolic state is generated by inputting wearable sensor data, lab test, and recorded patient data as input values to the model's function and parameters, and, together with values assigned to those parameters, determines a patient's metabolic health. As described herein, the term “model” refers to the result of the machine learning training process. Specifically, the model describes the generation of a function for representing a patient's metabolic state and the determined parameter values that the function incorporates. “Parameter values” describe the weight that is associated with at least one of the featured input values. “Input values” describe the variables of the function or the conditions to be used in conjunction with the parameter values to determine the risk score. Input values can be thought of as the numerical representations of the various features that the model takes into account, for example the input biosignals. During training, from input values of the training dataset, the parameter values of a model are derived. Further, the training data set is used to define the parameter values at a specified time interval, whereas the input values are continuously updated by the patient's conditions.

The digital twin module 350 may include a combination of machine-learning models to generate various representations of a metabolic state, for example metabolic models trained to predictively model a patient's metabolic state based on recorded nutrition data, medication data, symptom data and lifestyle data, and to model a patient's true metabolic state based on sensor data and lab test data. The digital twin module 350 may input patient data 320, for example nutrition data, medication data, symptom data, or lifestyle data, into combination metabolic models to predict a patient's metabolic state that would result from the recorded patient data. The digital twin module 350 may compare a recorded timeline of patient data (e.g., foods consumed by the patient, medications taken by the patient, and symptoms experienced by the patient) during a time period to a metabolic state generated for the time period to determine an effect of each food item, medication, and symptom on the metabolic state of the patient.

Additionally, the digital twin module 350 may implement one or more metabolic models to predict a patient's metabolic state that would result from the recommended nutrition, medication, or lifestyle changes included in a recommendation. Alternatively, the digital twin module 350 may receive biological data, for example sensor data and lab test data, as inputs to metabolic models to determine a patient's actual metabolic response to the patient data 320.

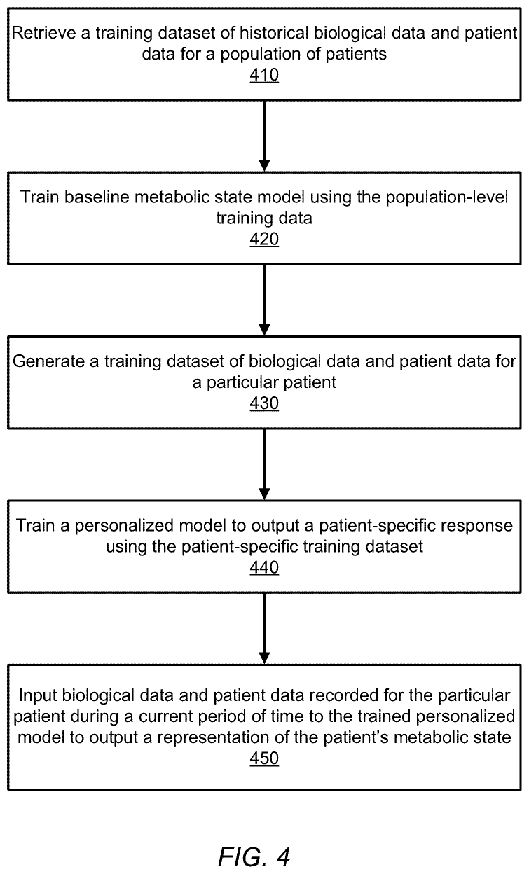

FIG. 4 is an illustration of the process for training a machine-learning model to output an aspect of a patient's metabolic health, according to one embodiment. The digital twin module 350 retrieves 410 a training dataset comprising historical biosignals (e.g., historical sensor data and lab test data) and patients measured and/or recorded for an entire population of patients. Each historical measurement of biological data and record of patient data is assigned a timestamp representing when the patient experienced the measurement/recording and a label identifying its impact on a patient's metabolic health, the patient's metabolic response to the measurement, or both. Using the training dataset of population-level data, the digital twin module 350 trains 420 a baseline model. The training dataset of population-level data comprises labeled metabolic states recorded for a population of patients and sensor data and lab test data that contributed to each labeled metabolic state. Once trained, the baseline model may be implemented to determine a metabolic state of a representative patient of the population of patients (e.g., an average patient) given a set of biological inputs, for example biological data or patient data.

In some implementations, the baseline model may be further trained to generate a personalized representation of a patient's metabolic health. In such implementations, the digital twin module 350 generates 430 an additional training dataset of biological data and patient data for a particular patient. The digital twin module 350 accesses both measured biological data and recorded patient data for a particular patient and aggregates that data into a training dataset. Similar to the historical training dataset, the biological data and patient data of the training dataset are assigned a timestamp and a label to characterize how each biological input impacts the particular patient's metabolic state. Using the training dataset of patient-specific data, the digital twin module 350 trains 440 a personalized metabolic model. Once training, biological data and patient data recorded during a subsequent period of time may be input 450 to the trained model to output a representation of a particular patient's metabolic state.

Depending on the type of data input to either the personalized or baseline metabolic model, the digital twin module 350 may generate a representation of a patient's true metabolic state or their predicted metabolic state. Biological data, for example data recorded by a wearable sensor or a lab test, may be input to a model to generate a representation of a patient's true metabolic state consistent with the description above. Alternatively, patient data, for example nutrition data, medication data, symptom data, and lifestyle data, may be input to a model to generate a prediction of patient's current metabolic state consistent with the description above.

Training both models in such a manner enables the patient health management platform 130 to predict a patient's metabolic response to future input stimuli (i.e., patient data 320 recorded by a patient in the future) for not just patients already included in the training dataset, but also new patients included in a holdout dataset because the model only relies on the knowledge representing a patient's current metabolic state and the patient's input stimuli to predict their patient-specific response. Additionally, the model predicts a patient's response to input stimuli for each patient at different stages of his or her treatment because the platform maintains a history of a patient's changing metabolic condition. Finally, it allows for long-range precision prediction of the patient's metabolic state by using current and short-range predictions to inform longer-range predictions.

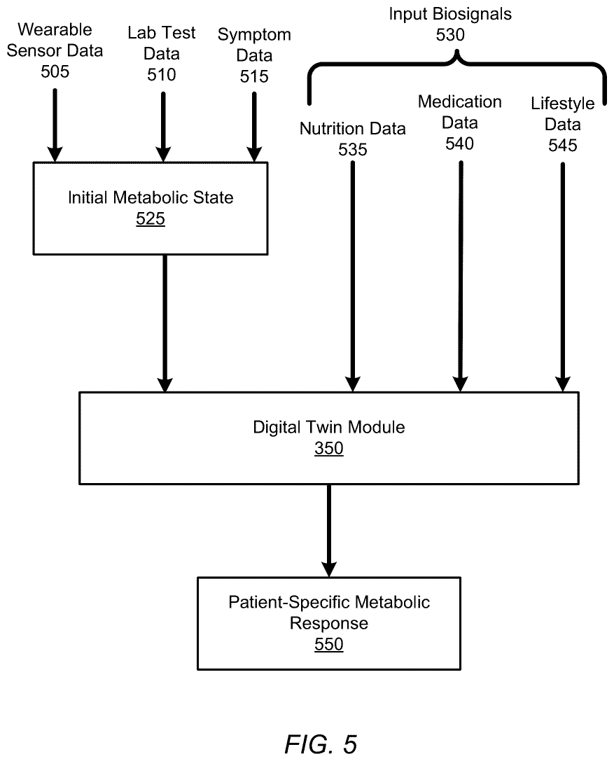

FIG. 5 is an illustration of the process for implementing a machine-learning model, according to one embodiment. For a given time period, biosignals recorded as wearable sensor data 505, lab test data 510, and symptom data 515 are representative of a patient's actual, current metabolic state. Accordingly, based on these input biosignals, the patient response module generates an initial metabolic state 525. When sufficient training data exists for a particular patient, the initial metabolic state 525 may be determined using a metabolic model(s). Alternatively, the initial metabolic state 525 may be determined using metabolic model(s) trained for a population of patients. Additionally, the digital twin module 350 relies on input biosignals 530, which represent biosignals that may impact a patient's metabolic state, either deteriorating or improving the state. For example, input biosignals 530 may include nutrition data 535, medication data 540, and lifestyle data 545 recorded for a patient at a time occurring after the generation of the initial metabolic state. In addition to the initial metabolic state 525, the digital twin module 350 receives the input biosignals 530 recorded by the patient as inputs one or more metabolic models. Accordingly, digital twin module 350 models the patient's patient-specific metabolic response 550 to the inputted biosignals. Described differently, the patient-specific metabolic response 550 represents one or more changes in a patient's initial metabolic state caused by, or at least correlated with, the input biosignals 530.

For a second time period following the determination of the patient-specific metabolic response 550, the platform 130 continues to record wearable sensor data 505, lab test data 510, and symptom 515. Given biosignals recorded as wearable sensor data 505 and lab test data 510 as inputs, the aggregated output of the combination of metabolic models (e.g., the true metabolic state) describes what a patient's metabolic response actually is during a time period. Given nutrition data 535, medication data 540, and lifestyle data 545 (e.g., input biosignals 530) recorded during the same time period as inputs, the aggregated output of the combination of metabolic models (e.g., the predicted metabolic state) describes what a patient's metabolic response should be during the time period. Accordingly, a comparison of the two outputs allows the platform 130 to verify the timeliness, accuracy, and completeness with which a patient recorded the input biosignals 530.

More information regarding the patient health management platform 130 and its components, as well as the interactions between those components, can be found in U.S. patent application Ser. No. 16/993,144, filed Aug. 13, 2020, U.S. patent application Ser. No. 16/992,184, filed Aug. 13, 2020, and U.S. patent application Ser. No. 16/993,189, filed Aug. 13, 2020, each of which are incorporated by reference herein in their entirety.

### IV.C Patient-Specific Recommendations

The recommendation module 360 offers short-term adaptiveness to the patient by providing multiple solutions for achieving the same short term health outcome. For example, the recommendation module 360 recommends an array of dietary or activity modifications to the patients with the same projected effect on the patient's metabolism. The recommendation module 360 generates patient-specific recommendations for improving the metabolic state of a patient. A patient-specific recommendation may include adjustments for a patient to make to their lifestyle, for example dietary changes, activity changes, any other suitable lifestyle adjustments, or a combination thereof. As described herein, dietary changes include instructions for a patient to consume a particular food item(s) or stop consuming a particular food item(s). As described herein, activity changes include instructions for a patient perform a particular activity(s) and when to perform such activities.

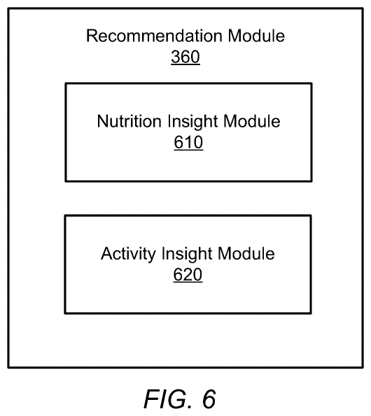

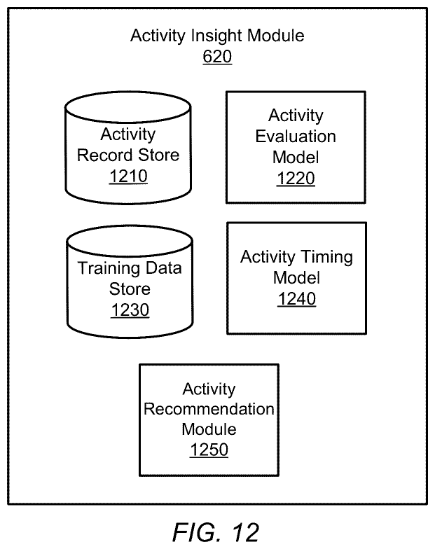

FIG. 6 is a block diagram of the system architecture of the recommendation module 360, according to one embodiment. The recommendation module 360 includes a nutrition insight module 610 and an activity insight module 620. However, in other embodiments, the recommendation module 360 may include different and/or additional components. The nutrition insight module 610 generates patient-specific insights regarding the effect of food items of interest to the patient on their metabolic state. In some embodiments, the nutrition insight module 610 generates patient-specific diet recommendations to be displayed to the patient. The nutrition insight module 620 is further described below with reference to FIGS. 7-11. The activity insight module 620 generates patient-specific insights regarding the effect of various physical activities on their metabolic state. In some embodiments, the activity insight module 620 generates patient-specific activity recommendations to be displayed to the patient. The activity insight module 620 is further described below with reference to FIGS. 12-15.

In some embodiments, the patient provides feedback and preferences to the recommendation module 360 and the recommendation module 360 may modify recommendations to fit the patient's preferences. By accounting for the patient's feedback and preferences, the recommendation module 360 increases the likelihood that a patient will adhere to the generated recommendation(s).

The nutrition insight module 610 classifies food items based on the predicted effect of consuming the food item on a patient's metabolic state. FIG. 7 is a block diagram of the system architecture of the nutrition insight module 610, according to one embodiment. The nutrition insight module 610 may include a nutrition data store 710, food classification model 720, training data store 730, and food recommendation module 740. However, in other embodiments, the nutrition insight module 610 may include different and/or additional components.

For a given patient, the nutrition data store 710 may retrieve and store a current metabolic profile of the patient. As described above, the patient's current metabolic profile includes biosignal measurements collected for the patient during a current time period and the current metabolic state of the patient determined based on the collected biosignal measurements and the techniques described above.

The nutrition data store 710 also stores a record of food items previously recorded and/or consumed by the patient. For example, if a patient records that they consumed a meal of salmon and rice, both salmon and rice are added to the record of food items for the patient. As new food items are recorded by the patient, the nutrition data store 710 updates the record of food items to reflect the newly recorded food items. The nutrition data store 710 stores the record of food items with a timestamp describing when the food item was recorded. In some embodiments, the nutrition data store 710 also accesses and stores nutrition data for each recorded food item from the nutrition database 140. For example, the nutrition data store 710 may store that a serving of salmon contains 20 g of protein per serving and 10 g of carbs. The nutrition data store 710 stores each recorded food item with a current classification of the food item describing an effect of the food item on a metabolic state of the patient.

In some embodiments, the metabolic state of a patient may refer to the condition of the patient's metabolism, encompassing processes like energy production, nutrient utilization, and waste elimination crucial for maintaining life. The metabolic state may vary depending on factors like nutritional intake, physical activity, and underlying health conditions. Different metabolic states include resting basal metabolic rate (BMR), fed and fasting states, ketosis characterized by increased ketone levels, and fluctuations in metabolic rate such as hypermetabolism or hypometabolism.

The effect of a food item on the metabolic state of a patient may vary based on several factors, including the nutritional composition of the food, the individual's metabolic health, and their overall dietary pattern. For example, food items containing carbohydrates, particularly those with high glycemic index (GI), can lead to rapid spikes in blood glucose levels after consumption. Such rapid spikes in blood glucose can trigger insulin release from the pancreas to help regulate blood sugar. High-carbohydrate foods may exacerbate blood sugar fluctuations and contribute to metabolic dysfunction in patients with impaired glucose tolerance or insulin resistance. Conversely, foods high in fiber, protein, and healthy fats can slow the absorption of glucose into the bloodstream, leading to more stable blood sugar levels.

In some embodiments, a single food item may have both positive and negative effects on a patient's metabolic state depending on factors including, but not limited to, nutrient composition, processing, individual metabolic response, and quantity consumed. For instance, nuts can offer benefits such as healthy fats and fiber, but excessive consumption may lead to weight gain. Similarly, whole fruits provide essential nutrients and fiber beneficial for metabolic health, but fruit juices can contain concentrated sugars and lack fiber that could potentially cause rapid spikes in blood sugar levels. Moreover, individual factors like genetic differences and gut microbiota composition may influence how a patient metabolizes certain nutrients. Therefore, the same food item may have different effects on different patients' metabolic states.

In some embodiments, a food item consumption may affect satiety of a patient. As described herein, satiety refers to a patient's feeling of fullness and a patient's satisfaction experienced after eating a meal or snack. Satiety is a physiological response to food intake that helps regulate energy balance and nutrient intake. Satiety plays a crucial role in regulating food intake and preventing overeating by influencing appetite and meal terminationby sending signals to the brain that one has had enough food to meet their immediate energy needs. Accordingly, satiety involves various metabolic processes, such as the digestion and absorption of nutrients, the release of hormones involved in appetite regulation (such as leptin and ghrelin), and the signaling pathways within the brain that control hunger and fullness. Several factors contribute to satiety, including the volume and composition of food consumed, nutrient content (such as protein, fiber, and fat), and the release of hormones involved in appetite regulation, such as leptin, ghrelin, and peptide YY. Foods that promote satiety tend to be high in protein, fiber, and water content, as they take longer to digest and can help delay hunger cues. In some cases, achieving satiety is essential for maintaining a healthy weight and overall healthy metabolic state. Additionally, individual factors, for example underlying metabolic health, sleep quality, and activity levels, influence satiety, so the same food item may result in different levels of satiety for different patients.

The nutrition data store 710 stores a classification assigned to a food item, which may indicate whether the patient can safely consume the food item or should avoid that food item based on the predicted effect of the food item on the patient's metabolic state. For example, foods high in sugar such as donuts, ice cream, and candy bars may be classified as likely detrimental to a patient's metabolic state while foods high in nutrients such as avocado and chicken may be classified as likely to improve the patient's metabolic state. As the classification of a food item changes with changes in the metabolic health of the patient, the nutrition data store 710 is updated with the updated classification.

In some embodiments, when a patient records a new food item (e.g., a food that did not previously exist in the record of food items), the new food item is initially stored within the nutrition data store 710 with a baseline classification. The baseline classification describes an expected effect of the food item based on data collected for a population of secondary patients. The nutrition insight module 610 identifies the population of secondary patients such that each secondary patient shares one or more qualities with the patient who recorded the food item. As described herein, a secondary patient may be demographically similar or metabolically similar to the patient who recorded the food item. The nutrition insight module 610 generates a baseline classification for the new food item based on the classification of the food item determined for the identified secondary patients. In one embodiment, nutrition insight module 610 determines the baseline classification as an average of the classifications determined for the secondary patients. In another embodiment, the nutrition insight module 610 determines the baseline classification as a weighted average of the classifications determined for the secondary patients where the weights correlate with a level of similarity between each secondary patient and the patient recording the food item.

The nutrition insight module 610 applies machine-learning techniques to classify food items based on their effect on a patient's metabolic state. The nutrition insight module 610 710 encodes the record of food items stored in the nutrition data store 710, the biosignal measurements collected during the current time period, and the current metabolic state of the patient into a feature vector. As described herein, a feature vector is a representation of the current metabolic state of the patient, which may be processed by a machine-learning model (e.g., the food classification module 720) to predict the effect of a food item on a patient's current metabolic state. Each feature vector may include values describing or characterizing the attributes (features) of the corresponding metabolic state. In some implementations, the feature vector may correspond to a data element in a multi-dimensional vector space, where each dimension represents a feature. Machine-learning algorithms operate within this multi-dimensional vector space to learn patterns and relationships among feature vectors and components of feature vectors. The exact composition of the feature vector may vary depending on the context and available data of the metabolic state. In some embodiments, the feature vector may include encoded features that correspond to one or more biosignal measurements, e.g., blood glucose level, body composition, resting metabolic rate, blood pressure, etc. The encoded feature vector is input to the food classification model 720, which predicts the effect of each recorded food item on the patient's current metabolic state and classifies the food item based on the predicted effect.

The food classification model 720 predicts the effect of each food item on the metabolic state of the patient by modeling the effect of the food item on one or more of the measured biosignals. The food classification model 720 may be a mathematical function or another more complex logical structure, trained using a combination of features stored in the training data store 730 to determine a set of parameter values stored in advance and used as part of the prediction analysis. To predict the effect of a food item on the metabolic state of a patient, the food classification model 720 models the effect of the food item on the response of one or more biosignals. In some embodiments, the effect of a food item is predicted/evaluated for a specific aspect of the metabolic state, e.g., satiety, weight, nutritional status, physical activeness, etc. and the food item is classified based on the effect of the food item. In some embodiments the effect of a food item and the classification of the food item for a patient is an overall evaluation/prediction on the entire metabolic state of the patient.

The food classification model 720 is trained using the training data store 730, which is made up of a large number of entries. Each training example, e.g., an entry of the training data store 730, includes a food item consumed by a patient and a metabolic state recorded for the patient when the food item was consumed. Each entry is assigned a label describing an effect of the food item on the recorded metabolic state and a classification describing the effect. In one or more embodiments, the training data store 730 includes patient data collected for a particular patient; the training data store 730 may only store foods consumed by the particular patient and metabolic states recorded and verified for that patient. In other embodiments, the training data store 730 includes food items consumed by any patient of a larger population of patients and metabolic states recorded for the population of patients. For example, if the amount of patient data stored within the training data store 730 for a particular patient does not satisfy a threshold amount, the nutrition insight module 610 may augment the training data store 730 with patient data collected for other patients.

As metabolic effects predicted by the food classification model 720 are verified by medical professionals monitoring a patient's metabolic health, the training data store 730 may be continuously updated with entries pertaining to newly recorded food items. Additionally, the training data store 730 may be periodically updated with entries of novel food items or novel features extracted from existing entries in the training data store 730. Accordingly, the food classification model 720 may be iteratively trained such that the food classification model 720 continues to learn and refine its parameter values based on the new and updated data set 730. Iteratively re-training the food classification model 720 in the manner discussed above allows the classification model 720 to more accurately predict the metabolic effects of a food item.

In some embodiments, the nutrition insight module 610 updates the food classification model 720 by using a loss value (e.g., by using a loss function, cost function, etc.). The loss value indicates how well the prediction matches the true label of a training example. To generate a loss value, the nutrition insight module 610 compares the predicted effect of a food item in the training data store 730 with the labeled effect for the food item. During training, the food classification model 720 determines parameter values for each feature of the patient's metabolic profile input to the model 720 by analyzing and recognizing correlations between the features associated with the metabolic profile and the metabolic effect of the food item. The food classification model 720 adjusts the model's parameters (e.g., weights in neural networks) to minimize the loss function. The food classification model 720 updates the parameters of the food classification model based on the loss value and the parameter values are stores the parameter values for later use in classifying food items.

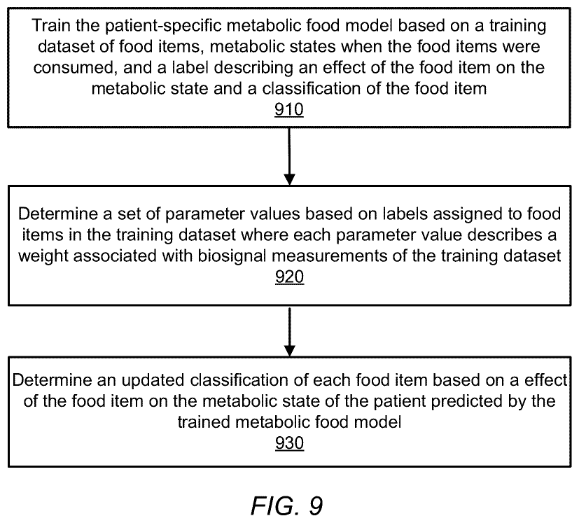

In some embodiments, the food classification model 720 may be trained on training data for individual food items or a particular category of food items to generate a baseline model for the food item or category of food items. Depending on the food item being evaluated, the nutrition insight module 610 may select a particular baseline model. For example, if the food item is a banana, the nutrition insight model 610 may select the baseline model for bananas or the category “fruits” and input the encoded feature vector to the selected baseline model. In such embodiments, the baseline model may be further trained using a particularized training data set comprising training data for the particular category of items. Accordingly, a baseline classification model may be further trained to predict a metabolic effect for a particular food item or category of food item. Training of the food classification model 720 is further described below with reference to FIG. 9.

Whether a patient's metabolic health is improving or not can be characterized by changes in biosignal measurements between time periods. For example, if a patient's average blood glucose levels significantly increase from one time period to another, the patient's metabolic health has likely worsened. Alternatively, if an obese patient's weight steadily declines from one time period to another, the patient's metabolic health has likely improved. As described above, the food classification model 720 predicts the effect of a food item on a patient's metabolic state by predicting fluctuations in biosignal measurements resulting from consuming the food item. Accordingly, the food classification model 720 classifies the food item by assigning a label describing the predicted effect of the food item on one or more biosignal measurements. In one embodiment, the food classification model 720 classifies the food item as improving a patient's metabolic state, worsening the patient's metabolic state, or having no effect on the patient's metabolic state. The food classification model 720 determines whether consumption of a food item will improve, worsen, or not impact the patient's metabolic state based on the predicted effect of the food item on the one or more measured biosignals. In one embodiment, the food classification model 720 classifies food items based on a blood glucose response predicted if the patient were to consume a food item.

In one implementation, the food classification model 720 may classify a food item based the effect of the food item on a patient's satiety which is reflected by the one or more measured biosignals. In some embodiments, the food classification model 720 may classify a food item based on a single effect of the food item on the metabolic state of the patient. Alternatively, the food classification model 720 may classify a food item based on a combined effect (e.g., combinations of the biosignals) of the food item on the metabolic state of the patient. In some embodiments, the food classification generates a personalized classification for a specific patient, for example, considering the patient's genetic differences, medical history, etc.

In one example, changes in daily calories consumed can be used to assess the satiety-inducing effects of different food items. A patient's calories consumed may be tracked across all the meals for the day. The patient may consume a food item or meal containing the food item which may be used to characterize how satiating the meal was for the patient. As an example, a patient may consume either a protein-rich meal (such as grilled chicken breast with steamed vegetables) or a carbohydrate-rich meal (such as white bread with jam), and the patient's calories consumed may be tracked across all the meals for the day. After consuming the protein-rich meal, the patient may experience a decrease in ghrelin levels, which corresponds to a decrease in hunger and an increase in satiety. In contrast, after consuming the carbohydrate-rich meal, the patient may experience a smaller decrease in ghrelin levels compared to the protein-rich meal, which suggests that the carbohydrate-rich meal may not be as effective at suppressing hunger and inducing satiety. As a result, the patient ends up consuming a larger number of calories for the rest of the day to compensate for the hunger. Accordingly, the difference in daily calories consumed between the protein-rich and carbohydrate-rich meals indicates that the protein-rich meal is more satiating. For example, the slower digestion and absorption of protein and the release of satiety hormones like peptide YY may contribute to prolonged feelings of fullness and reduced hunger. In this case, the food classification model 720 may determine that the protein-rich food item has a healthier effect to the patient's metabolic state compared to the carbohydrate-rich food item.

Accordingly, in some embodiments, the food classification model 720 may use the ghrelin level as one of the biosignals to reflect the patient's metabolic state, e.g., satiety. The food classification model 720 may be trained to classify a food item based on its effect on a patient's ghrelin level. For example, the calories included in a food item may be selected as a feature for training the food classification model 720. The ghrelin level may be collected before/after a patient consumes the food item. The food classification model 720 is trained to predict the ghrelin level change due to the consumption of the food item. Based on the predicted values/changes of the ghrelin level before and after consuming the food item, the food classification model 720 classifies a food item, e.g., risky, healthy, etc.

In some implementations, the food classification model 720 may be trained to predict a patient's metabolic state, such as, a satiety level based on the ghrelin level. For example, the food classification model 720 may access a training dataset including a plurality of training example. Each example may include a ghrelin level of a patient and an associated indication of satiety of the patient. The satiety of the patient may be indicated/determined from a patient's input, and/or may be determined using one or more biosignals. The food classification model 720 is trained with the training database to predict a patient's satiety level based on the patient's ghrelin level.

In some implementations, the food classification model 720 may include one or more models that are trained to classify a food item based on the predicted metabolic state, e.g., satiety level of the patient. For example, the food classification model 720 may access a training dataset including a plurality of training example. In some examples, each example may include calories intake for a patient consuming a food item, one or more biosignals including ghrelin level of the patient, and an associated indication of satiety of the patient. Calories included in a food item may be determined using a lookup table. Bisosignals, such as, ghrelin level may be collected before/after a patient consumes the food item. The satiety of the patient may be indicated/determined from a patient's input, and/or may be determined using one or more biosignals. The food classification model 720 is trained to predict the patient's satiety level due to the consumption of the food item. Based on the predicted values/changes of the metabolic state, e.g., satiety level for consuming the food item, the food classification model 720 classifies a food item, e.g., risky, healthy, etc.

In another example, changes in glucose levels can be used as to assess the satiety-inducing effects of different food items. A patient's glucose levels may be measured before and after the patient consumes a food item or meal containing the food item to characterize how satiating the meal was for the patient. As an example, a patient may consume either a high-fiber, whole grain meal (such as quinoa salad with mixed vegetables) or a meal high in refined carbohydrates (such as white rice with sweetened sauce). After consuming the high-fiber, whole grain meal, the patient may experience a gradual increase in glucose levels followed by a slower decline. This gradual rise and fall in glucose levels indicate a steady release of carbohydrates into the bloodstream, leading to sustained energy levels and prolonged satiety. In contrast, after consuming the meal high in refined carbohydrates, the patient may experience a rapid increase in glucose levels, followed by a sharp decline. This rapid spike and subsequent crash in glucose levels can lead to feelings of hunger and decreased satiety shortly after eating. Accordingly, the difference in postprandial glucose responses between the two meals highlights the importance of carbohydrate quality in regulating satiety. The high-fiber, whole grain meal with slower-digesting carbohydrates leads to more stable glucose levels and sustained feelings of fullness, whereas the meal high in refined carbohydrates results in rapid fluctuations in glucose levels and reduced satiety. In this case, the food classification model 720 may determine that the high-fiber, whole grain food item has a healthier effect to the patient's metabolic state compared to the refined carbohydrates food item.

In some embodiments, the food classification model 720 may use the glucose level as one of the biosignals to reflect the patient's metabolic state, e.g., satiety. The food classification model 720 may be trained to classify a food item based on its effect on a patient's glucose level and/or glucose response to the consumption of the food item. For example, the calories/nutrition content included in a food item may be selected as a feature for training the food classification model 720. The glucose level may be collected before/after a patient consumes the food item. In one example, the food classification model 720 is trained to predict the glucose response due to the consumption of the food item. Based on the predicted values/changes of the glucose level before and after consuming the food item, the food classification model 720 classifies a food item, e.g., risky, healthy, etc.

In some implementations, the food classification model 720 may be trained to predict a patient's metabolic state, e.g., satiety level, based on the glucose level. For example, the food classification model 720 may access a training dataset including a plurality of training example. Each example may include a glucose level of a patient and an associated indication of satiety of the patient. The satiety of the patient may be indicated/determined from a patient's input, and/or may be determined using one or more biosignals. The food classification model 720 is trained with the training database to predict a patient's satiety level based on the patient's glucose level.

In some implementations, the food classification model 720 may include one or more models that are trained to classify a food item based on the predicted metabolic state, e.g., satiety level of the patient. For example, the food classification model 720 may access a training dataset including a plurality of training example. In some examples, each example may include the calories intake for a patient consuming a food item, one or more biosignals including a glucose level of the patient, and an associated indication of satiety of the patient. Calories included in a food item may be determined using a lookup table. Bisosignals, such as, glucose level (and/or ghrelin level) may be collected before/after a patient consumes the food item. The satiety of the patient may be indicated/determined from a patient's input, and/or may be determined using one or more biosignals. The food classification model 720 is trained to predict the patient's satiety level due to the consumption of the food item. Based on the predicted values/changes of the metabolic state, e.g., satiety level, for consuming the food item, the food classification model 720 classifies a food item, e.g., risky, healthy, etc.

The food classification model 720 may classify a food item based on various criteria, for example the one or more biosignals that are used to evaluate the effect of the food item to the metabolic state, and/or a specific aspect of the metabolic state. The food classification model 720 may classify a food item based on the numerical values of the corresponding biosignals. In one embodiment, the food classification module 720 classifies a food item based on the absolute value measured for the corresponding biosignal(s). In another embodiment, the food classification module 720 classifies a food item based on a range of measurements corresponding to the measured value of a biosignal(s), such as high, medium, or low, or a specific range corresponding to different physiological states or conditions. In other embodiments, the food classification model 720 may classify (or update the classification of) a food item based on the absolute value of the corresponding biosignals. In some embodiments, the food classification model 720 may classify a food item based on changes in the values of the corresponding biosignals. The food classification model 720 may additionally classify a food item based on the biosignal's statistical properties such as mean, variance, skewness, or kurtosis; or based on recognizable patterns or features extracted using machine learning algorithms, pattern recognition techniques, or expert knowledge, and the like.

The nutrition insight module 610 may implement the food classification model 720 in response to one or more trigger conditions. In one embodiment, the food classification model 720 generates classifications for food items in the nutrition data store 710 periodically or at the conclusion of a time interval (e.g., every week, every month, etc.). In another embodiment, the food classification model 720 generates classifications for food items in response to a request from a user, for example a medical provider, an authorized caretaker, or the patient themselves. In another embodiment, the food classification model 720 generates classifications for food items in response to receiving a new entry of patient data. In one embodiment, the food classification model 720 generates a classification for each food item in the nutrition data store. In another embodiment, the food classification model 720 generates a classification for only food items in a patient's record of food items (e.g., only food items previously consumed by the patient or that the patient has expressed interest in consuming). The nutrition data store 710 stores the most recent classification generated by the food classification model 720 for each food item, so the classifications generated in response to a triggering condition are referred to herein as “updated classifications.”

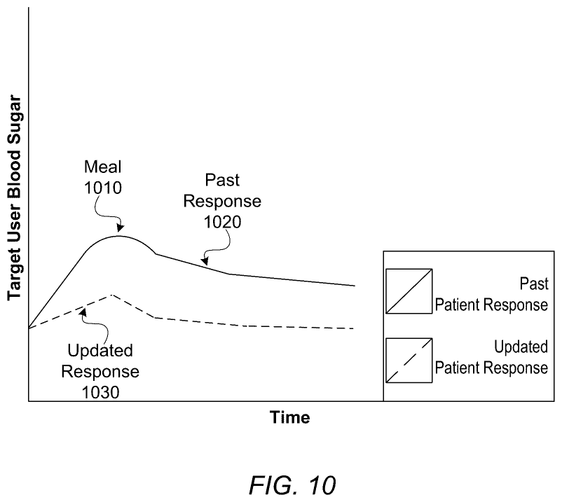

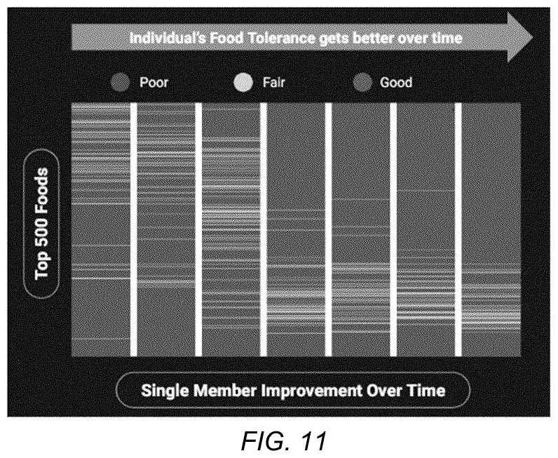

The food recommendation module 740 provides insights generated by the food classification model 720 to a patient, for example in the form of a notification, a recommendation or both. The food recommendation module 740 generates and transmits the notification to the patient. The food record model 740 may generate a notification displaying the updated classifications for each food item of the patient's record of food items. For example, the notification may code the classifications using a color scheme where a first color (e.g., green) identifies food items that will improve a patient's metabolic state, a second color (e.g., yellow) identifies food items that will not affect a patient's metabolic state, and a third color (e.g., red) identifies food items that will worsen a patient's metabolic state. In some embodiments, the food recommendation module 740 generates a notification displaying particular food items whose classification has changed between the current time period and a previous time period. In such embodiments, the food recommendation module 740 compares the updated classification for each food item with the previous classification for the food item and identifies a subset of the recorded food items where the updated classification differs from the current classification. The notification may also include a recommendation for the patient to consume food items of the identified subset classified as improving the metabolic state of the patient. Example notifications generated by the food recommendation module 740 are further described below with reference to FIGS. 10 and 11.

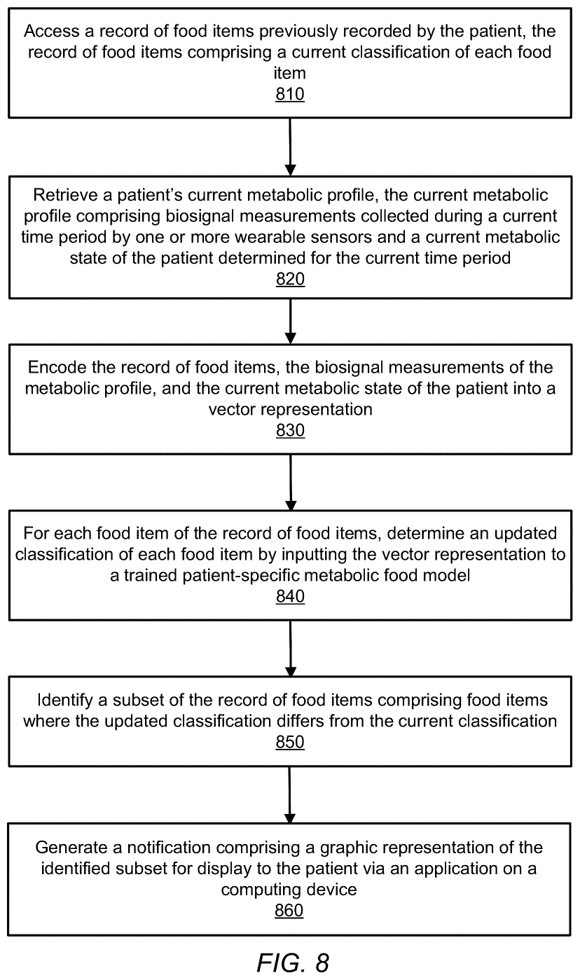

FIG. 8 is a flowchart illustrating a process for generating a recommendation for a patient with classifications of food items generated by a metabolic food model, according to one embodiment. The nutrition insight module 610 accesses 810 a record of food items previously recorded by a patient. The record of food items includes a current classification of each food item. The classification of each food item describes an effect of the food item on a metabolic state of the patient. The nutrition insight module 610 retrieve 820 a patient's current metabolic profile, which includes biosignal measurements collected during a current time period by one or more wearable sensors and a current metabolic state of the patient determined for the current time period.

The nutrition insight module 610 encodes 830 the record of food items, the biosignal measurements of the metabolic profile, and the current metabolic state of the patient into a vector representation to be input to a patient-specific metabolic food model (e.g., the food classification model 720). For each food item in the record of food items, the nutrition insight module 610 determines 840 an updated classification of each food item by inputting the vector representation to the patient-specific metabolic food model.

The nutrition insight module 610 identifies 850 a subset of food items of the record of foods items where the updated classification differs from the current classification. The nutrition insight module 610 generates 860 a notification comprising a graphic representation of the identified subset of food items for display to the patient via an application on a computing device. The notification may include a recommendation for the patient to consume food items of the identified subset classified as improving the metabolic state of the patient.

FIG. 9 is a flowchart illustrating a process for training a metabolic food model, according to one embodiment. The nutrition insight module 610 trains 910 the patient-specific metabolic food model based on a training dataset of food items. For each food item entry, the training data set includes a metabolic state when the food item was consumed and a label describing an effect of the food item on the metabolic state and a classification of the food item. During training, the nutrition insight module 610 may determine 920 a set of parameter values based on labels assigned to food items in the training dataset where each parameter value describes a weight associated with biosignal measurements of the dataset. The nutrition insight module 610 determines 930 an updated classification of each food item based on an effect of the food item on the metabolic state of the patient predicted by the trained metabolic food model.

FIG. 10 is a graphic illustrating changes in a user's responses to a food item over time, according to one embodiment. The graphic illustrates a patient's metabolic response to consuming a meal 1010 over a previous time period and a current time period. During the previous time period, the patient experienced the past metabolic response 1020-a significant spike in blood sugar after consuming the meal 1010 (e.g., a sharp increase and decrease in the value of the blood sugar in a short period of time after the consumption of the meal 1010). During the current time period, the patient experiences the updated response 1030-a lower spike in blood sugar after consuming the same meal 1010 (e.g., a smaller increase in the value of the blood sugar in a longer period of time after the consumption of the meal 1030). In such circumstances, the food classification model 720 classified the meal 1010 as harmful to the patient's metabolic health during the past time period based on the patient's past response 1020. However, the food classification model 720 updates the classification of the meal 1010 during the current time period based on the updated response 1030.

FIG. 11 is a graphic illustrating changes in classifications of food items determined for a user over time, according to one embodiment. The illustrated graphic displays changes in the classifications of a patient's top 500 foods over seven subsequent time periods. A patient's top 500 foods may be selected by the patient themselves or based on the patient's record of food items. In some embodiments, the food recommendation module 740 may transmit the graphic illustration to the patient for display. For example, the graphic illustration may be displayed in a user interface with interactable user interface elements. The user may interact with the graphic illustration, such as, selecting a time period, number of food items to be displayed/interacted with, etc. In some embodiments, the user may select a specific food item in the graphic illustration and obtain its corresponding biosignals information and metabolic state information. For example, the user may select a specific food item in the graphic illustration, and the user interface may display the user's responses to a food item as shown in FIG. 10.

The activity insight module 620 identifies physical activities that will improve the metabolic state of the patient if the patient performs the physical activity. FIG. 12 is a block diagram of the system architecture for the activity insight module 620, according to one embodiment. The activity insight module 620 may include an activity record store 1210, an activity evaluation model 1220, a training data store 1230, an activity timing model 1240, and an activity recommendation model 1250. However, in other embodiments, the activity insight module 620 may include different and/or additional components.

For a given patient, the activity record store 1210 may retrieve and store a current metabolic profile of the patient. As described above, the patient's current metabolic profile includes biosignal measurements collected for the patient during a current time period and the current metabolic state of the patient determined based on the collected biosignal measurements and the techniques described above.

The activity record store 1210 also stores a record of activities previously recorded and/or performed by the patient. Examples of physical activities include, but are not limited to, walking, running, swimming, cycling, and such. For example, if a patient records that they went on a 30 minute walk, walking is added to the record of activities for the patient with a timestamp noting when the user went for the walk. As new activities are recorded by the patient, the activity record store 1210 updates the record of activities to reflect the newly recorded activities. The activity data store 1210 stores the record of activities with a timestamp noting when the activity was performed and/or recorded. The activity data store 120 may additional store information associated with each recorded activity including, but not limited to, a duration of the activity and one or more biosignal measurements collected for the patient during the activity, for example an average heart rate of the patient during the activity, an initial blood glucose level of the patient while performing the exercise, the patient's blood glucose level before the exercise, the time of day that the patient performed the exercise, and the patient's average blood glucose over 24 hours, and the patient's age.

The activity data store 1210 stores each recorded activity with a classification describing an effect of the activity on a metabolic state of the patient. In one embodiment, each recorded activity may be assigned a label indicating whether performing the physical activity will improve, worsen, or not affect the metabolic state of the patient. For example, activity that cause too high of a spike in a patient's blood glucose may be classified as detrimental to a patient's metabolic state compared to activities that do not spike the patient's blood glucose. As the classification for an activity change with changes in the metabolic health of the patient, the activity record store 1210 stores the updated classification.

Physical activities may have various effects on the metabolic state of a patient, depending on type, intensity, duration, and frequency, etc. of the physical activity. For example, a physical activity may increase energy expenditure through muscle engagement and elevated metabolic rate, aiding in calorie burning and utilization of stored energy sources. In another example, a weight of the patient may be used to indicate the patient's metabolic state. As another example, a physical activity may increase glucose regulation by enhancing insulin sensitivity and promoting glucose uptake by muscles, thereby stabilizing blood glucose levels and reducing the risk of insulin resistance and type 2 diabetes.

In some embodiments, the effect of a physical activity on a patient's metabolic state may be evaluated by changes in biosignal measurements between time periods. In one embodiment, the activity insight module 620 uses blood glucose monitoring as a direct and immediate method for assessing the effect of a 30-minute walk on a patient's blood sugar levels. For example, a baseline blood glucose level measurement may be collected before the patient begins their walk and a post-exercise blood glucose level measurement may be collected after the patient engages in their walk or at periodic intervals during the walk. This approach could also be applied prior to and after the patient engages in any other suitable activity, for example a 30-minute moderately intense exercise session. By comparing post-exercise blood glucose levels to baseline values, the activity evaluation module 1220 models the effects of the walk on blood sugar regulation. In some embodiments, the activity evaluation module 1220 interprets hypoglycemic responses, characterized by lower post-exercise blood glucose levels compared to baseline, as indicative of enhanced glucose utilization by muscles during exercise and interprets neutral or hyperglycemic responses as indicative of factors such as stress-induced cortisol release or insufficient exercise intensity to significantly impact blood sugar levels. In some embodiments, long-term monitoring through regular blood glucose assessments may be used to track the trends over time and tailor treatment plans accordingly.

In some embodiments, the activity evaluation model 1220 may use the blood glucose level as one of the biosignals to reflect the patient's metabolic state. The activity evaluation model 1220 may be trained to classify a physical activity based on its effect on a patient's blood glucose level and/or blood glucose response to performing the physical activity. For example, the time and/or strenuous level of the physical activity may be selected as a feature for training the activity evaluation model 1220. In some embodiments, the physical activity may be characterized by the calories burned for performing the physical activity. In some embodiments, the calories cost during performing the physical activity may be determined by using a lookup table. The blood glucose level may be collected before/after a patient performs the physical activity. In one example, the activity evaluation model 1220 is trained to predict the blood glucose response due to the physical activity. Based on the predicted values/changes of the blood glucose level before and after performing the activity, the activity evaluation model 1220 classifies a physical activity, e.g., risky, healthy, etc.

In some embodiments, the activity evaluation model 1220 may use the body weight as one of the biosignals to reflect the patient's metabolic state. The activity evaluation model 1220 may be trained to classify a physical activity based on its effect on a patient's body weight and/or changes of body weight for performing the physical activity. For example, the time and/or strenuous level of the physical activity may be selected as a feature for training the activity evaluation model 1220. In some embodiments, the physical activity may be characterized by the calories burned for performing the physical activity. In some embodiments, the calories cost during performing the physical activity may be determined by using a lookup table. The body weight may be collected periodically during a time period when a patient performs the physical activity. In one example, the activity evaluation model 1220 is trained to predict the body weight change due to the physical activity. Based on the predicted values/changes of the body weight, the activity evaluation model 1220 classifies the physical activity, e.g., risky, healthy, etc.

In some implementations, the activity evaluation model 1220 may be trained to predict a patient's metabolic state based on the blood glucose level and/or body weight. For example, the activity evaluation model 1220 may access a training dataset including a plurality of training example. Each example may include a blood glucose level/body weight of a patient and a metabolic state of the patient. The metabolic state of the patient may be indicated/determined from a patient's input, profile records, and/or may be determined using one or more biosignals. The activity evaluation model 1220 is trained with the training database to predict a patient's metabolic state based on the patient's blood glucose level/body weight.

In some implementations, the activity evaluation model 1220 may include one or more models that are trained to classify a physical activity based on the predicted metabolic state of the patient. For example, the activity evaluation model 1220 may access a training dataset including a plurality of training example. In some examples, each example may include the physical activity performed by the patient, one or more biosignals including a glucose level/body weight of the patient, and a metabolic state of the patient. The physical activity may be characterized by its performance time, strenuous level, etc. In some embodiments, the physical activity may be characterized by the calories burned for performing the physical activity. In some embodiments, the calories cost during performing the physical activity may be determined by using a lookup table. Bisosignals, such as, blood glucose level, body weight, etc., may be collected before/after a patient performs the physical activity, or periodically during the time period when the patient performs the physical activity. The metabolic state of the patient may be indicated/determined from a patient's input, and/or may be determined using one or more biosignals. The activity evaluation model 1220 is trained to predict the patient's metabolic state due to the performance of the physical activity. Based on the predicted values/changes of the metabolic state for performing the physical activity, the activity evaluation model 1220 classifies the physical activity, e.g., risky, healthy, etc.

In some embodiments, when a patient records a new activity (e.g., an activity that did not previously exist in the record of activities), the new activity is initially stored within the activity data store 1210 with a baseline classification describing an expected metabolic effect of the activity based on data collected for a population of secondary patients. The activity insight module 620 identifies the population of secondary patient such that each secondary patient shares one or more qualities with the patient who recorded the activity. As described herein a secondary patient may be demographically similar or metabolically similar to the patient who recorded the activity. The activity insight module 1210 generates a baseline classification for the new activity based on the classification of the activity determined for identified secondary patients. In one embodiment, the baseline classification is determined as an average of the classifications determined for the secondary patients. In another embodiment, the baseline classification is determined as a weighted average determined for the secondary patients where the weights correlate with how similar each secondary patient is the patient recording the activity.

The activity insight module 1210 applies machine-learning techniques to classify activities based on their effect on a patient's metabolic state. The activity insight module 620 encodes the record of activities stored in the activity record store 1210, the biosignal measurements collected during the current time period, and the current metabolic state of the patient into a feature vector. As described herein, a feature vector is a representation of the current metabolic state of the patient, which may be processed by a machine-learning model (e.g., the activity evaluation model 1220) to predict the effect of a given activity on a patient's current metabolic state.

The encoded feature vector is input to the activity evaluation model 1220, which predicts the effect of each recorded activity on the patient's current metabolic state and classifies the activity based on the predicted effect. The activity evaluation model 1220 predicts the effect of each activity on the metabolic state of the patient by modeling the effect of the activity on one or more measured biosignals. Similar to the food classification model 720 described above, the activity evaluation model 1220 may be a mathematical function or another more complex logical structure trained using a combination of features stored in the training data store 730. To predict the effect of an activity on the metabolic state of a patient, the activity evaluation model 1220 models the effect of an activity on the response of one or more biosignals. Consistent with the above description of the food insight module 610, the activity insight module 620 may implement the activity evaluation model 1220 in response to one or more trigger conditions.

The activity evaluation model 1220 is trained using the training data store 1230, which is made up of large number of entries. Each entry of the training data store 1230 includes an activity performed by a patient and a metabolic state recorded for the patient when the activity was performed. Each entry is assigned a label describing an effect of the activity on the recorded metabolic state and a classification characterizing the effect. In one or more embodiments, the training data store 1230 includes patient data collected for a particular patient; the training data store 1230 may only store activities performed by the particular patient and metabolic states recorded and verified for that patient. In other embodiments, the training data store 730 includes activities performed by any patient of a larger population of patients and metabolic states recorded for the population of patients. For example, if the amount of patient data stored within the training data store 1230 for a particular patients does not satisfy a threshold amount, the activity insight module 620 may augment training data store 730 with patient data collected for other patients.

As metabolic effects predicted by the activity evaluation model 1220 are verified by medical professionals monitoring a patient's metabolic health, the training data store 1230 may be continuously updated with entries pertaining to newly recorded activities. Additionally, the training data store 1230 may be periodically updated with entries of novel activities or novel features extracted from existing entries in the training data store 1230. Accordingly, the activity evaluation model 1220 may be iteratively such that the model 1220 continues to learn and refine its parameter values based on the new and updated data set 1230. Iteratively re-training the activity evaluation model 1220 in the manner discussed above allows the model 1220 to more accurately predict the metabolic effects of an activity.

To generate a loss value, the activity insight module 620 compares the predicted effect of an activity in the training data store 730 with the labeled effect for the activity. During training, the activity evaluation model 1220 determines parameter values for each feature of the patient's metabolic profile input to the model 1220 by analyzing and recognizing correlations between the features associated with the metabolic profile and the metabolic effect of the activity. The activity evaluation model 1220 updates the parameters of the activity classification model based on the loss and the parameter values are stores the parameter values for later use in classifying activities.

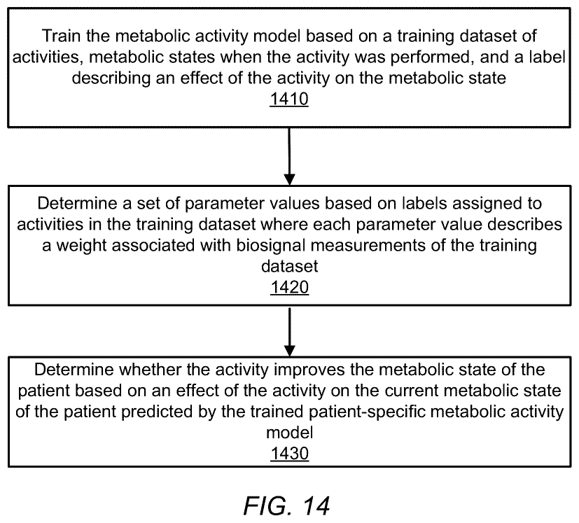

In some embodiments, the activity evaluation model 1220 may be trained on training data for individual activities or a particular category of activities to generate a baseline model for the activity or category of activities. Depending on the activity being evaluated, the activity insight module 620 may select a particular baseline model. For example, if the activity is walking, the activity insight model 620 may select the baseline model for walking or the category “low intensity activities” and input the encoded feature vector to the selected baseline model. In such embodiments, the baseline model may be further trained using a particularized training data set comprising training data for the particular category of items. Accordingly, a baseline classification model may be further trained to predict a metabolic effect for a particular activity or category of activities. Training of the activity evaluation model 1220 is further described below with reference to FIG. 14.

As described above, changes in a patient's metabolic health can be characterized by changes in biosignal measurements between time periods. Accordingly, the activity evaluation model 1220 predicts the effect of an activity on a patient's metabolic state by predicting fluctuations in biosignal measurements resulting from performance of an activity. Accordingly, the activity evaluation model 1220 classifies the activity by assigning a label describing the predicted effect of the activity on one or more biosignal measurements. In one embodiment, the activity evaluation model 1220 classifies the activity as improving a patient's metabolic state, worsening the patient's metabolic state, or having no effect on the patient's metabolic state. The activity evaluation model 1220 determines whether performance of the activity will improve, worsen, or not impact the patient's metabolic state based on the predicted effect of the activity on the one or more measured biosignals. In one embodiment, the activity evaluation model 1220 classifies activities based on a predicted blood glucose response if the patient were to perform the activity. The activity evaluation model 1220 may predict an average decrease in blood glucose over a time period following the patient performing the exercise. For example, the activity evaluation model 1220 may predict that the patient's blood glucose will decrease by 20% over the three hours following a run.

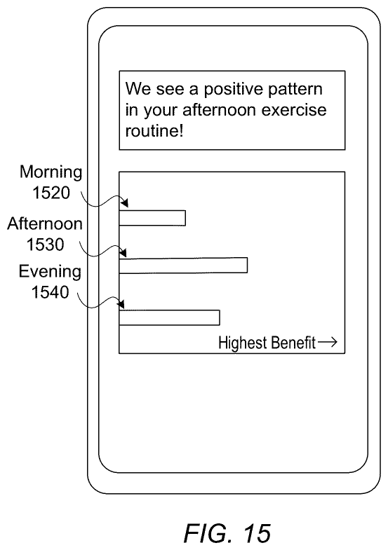

The activity recommendation module 1250 provides insights generated by the activity evaluation model 1220 to a patient, for example in the form of a notification, a recommendation or both. The activity recommendation module 1250 generates and transmits the notification to the patient. Consistent with the above description of the food recommendation module 750, the activity recommendation module 1250 may generate a notification displaying updated classifications for each activity of patients record of food items. In one embodiment, the activity recommendation module 1250 generates a notification displaying particular activities whose classification has changed between the current time period and a previous time period. In such embodiments, the activity recommendation module 1250 compares the updated classification for each activity with the previous classification for the activity and identifies a subset of the recorded activities where the updated classification differs from the current classification. In another embodiment, the activity recommendation module 1250 identifies a subset of the recorded activities predicted to either improve or maintain the metabolic state of the patient based on the classification determined by the activity evaluation model 1220. In such embodiments, the activity recommendation module 1250 generates a notification displaying the identified subset of activities to the patient with a recommendation for the patient to perform the activities of the identified subset. An example notification generated by the activity recommendation module 1250 is further described below with reference to FIG. 15.

Given the unique nature of metabolic health, some patients may be more susceptible to metabolic responses depending on the time of day when a patient performs a physical activity. Accordingly, in some embodiments, the activity timing model 1240 predicts the effect an activity at various times during the day. In such embodiments, each entry in the training data store 1230 includes a time when the corresponding activity was performed by a patient. Activity insight module 620 updates the encoded feature vector with a timestamp noting when each recorded activity was performed by the patient and inputs the updated feature vector to the activity timing model 1240. Consistent with the activity evaluation model 1220, the activity timing model 1240 may be a mathematical function trained using the combination of features in the training data store including the timestamp noting when the recorded activity was performed. To predict the effect of an activity on the metabolic state of a patient at different times, the activity timing model 1240 models the effect of the activity on the response of one or more biosignals as a function of time.

In some embodiments, the activity timing model 1240 generates predictions at varying levels of granularity based on the timestamps stored in the training data store 1230. In one embodiment, the activity timing model 1240 predicts the effect of an activity on an hourly basis (e.g., performing the activity at 9:00 AM versus 1:00 PM). In another embodiment, the activity timing model 1240 predicts the effect of an activity for different periods during the day (e.g., morning, afternoon, evening, night). In another embodiments, the activity timing model 1240 predicts an effect of an activity relative to other daily events (e.g., pre-meal or post-meal).

The activity timing model 1240 is trained using the training data store 1230 using the techniques described above with reference to the activity evaluation model 1220.

Based on the predictions the generated by the activity timing model 1240, the model 1240 identifies one or more timings correlated with the most significant improvements in the metabolic state of the patient or timings most likely to affect significant improvements in the metabolic state of the patient. In some embodiments, the activity recommendation module 1250 generates a notification with a recommendation for patient to perform one or more of the activities identified by the activity evaluation model 1220 at one or more times determined by the activity timing model 1240.

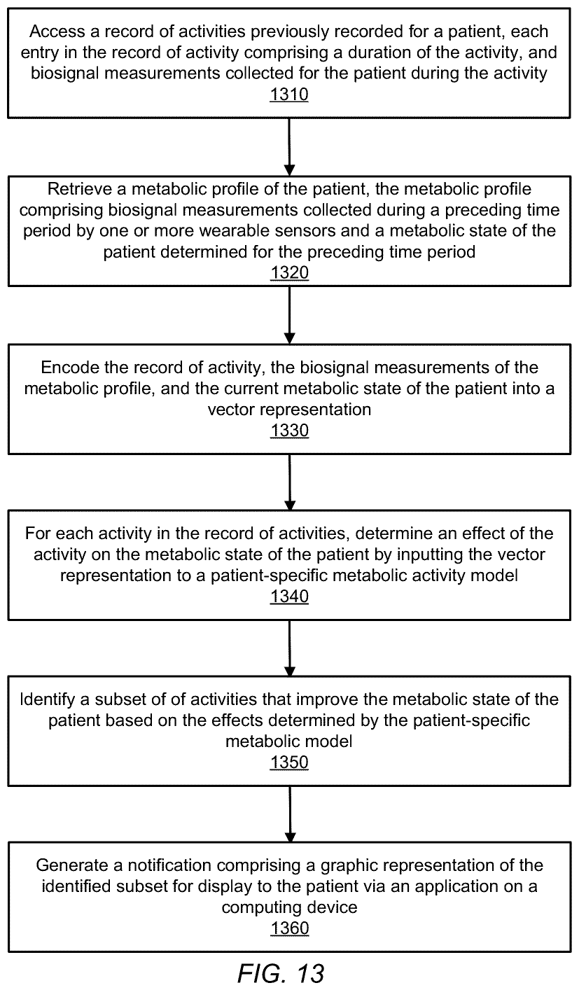

FIG. 13 is a flowchart illustrating a process for generating a notification for a patient with activities recommended by a metabolic activity model, according to one embodiment. The activity insight module 620 accesses 1310 a record of activities previously recorded for a patient. Each entry in the record of activity includes a duration of the activity and biosignal measurements collected for the patient during the activity. The activity insight module 620 retrieves 1320 a metabolic profile of the patient, which includes biosignal measurements collected during a preceding time period by one or more wearable sensors and a metabolic state of the patient determined for the preceding time period. The activity insight module 620 encodes 1330 the record of activity, the biosignal measurements of the current metabolic profile, and the current metabolic state of the patient into a vector representation.

The activity insight module 620 determines 1340 an effect of the activity on the metabolic state of the patient by inputting the vector representation to a trained patient-specific metabolic activity model (e.g., the activity evaluation model 1220). The activity insight module 620 identifies 1350 a subset of activities that improve the metabolic state of the patient based on the effects predicted by the patient-specific metabolic model. The activity insight module 620 generates 1360 a notification comprising a graphic representation of the identified subset of activities for display to the patient via an application on a computing device, for example the graphic illustrated in FIG. 15.

FIG. 14 is a flowchart illustrating a process for training a metabolic activity model, according to one embodiment. The activity insight module 620 trains 1410 the patient-specific metabolic activity model based on a training dataset of activities, metabolic states when the activity was performed, and a label describing an effect of the activity on the patient's metabolic state. The activity insight module 620 determines 1430 whether the activity improves the metabolic state of the patient based on an effect of the activity on the current metabolic state of the patient predicted by the trained patient-specific metabolic activity model.

FIG. 15 is an illustration of a notification recommending a patient perform activities in the afternoon, according to one embodiment. As illustrated in FIG. 15, the activity insight module 620 may generate a notification with a recommendation for the patient to perform one or more activities. The illustrated notification may contain a graphic display including times of day when the patient could perform the activity, for example morning 1520, afternoon 1530, and evening 1540, and a benefit of performing the activity at each time. The notification further contains a message identifying particular times of day predicted to have the most positive effect if the patient were to perform the activity. The notification may be displayed in a graphic user interface with interactive user interface elements. The user may interact with the graphic illustration, for example by selecting a time period, activities to be displayed/input, etc. In some embodiments, the user may select a specific activity in the graphic illustration and obtain its corresponding biosignals information and metabolic state information. For example, the user may select a specific activity in the user interface, and the user interface may display the user's responses to the activity and/or the trend of the response to the activity over time.

In some embodiments, the recommendation module 360 may evaluate the combined effect on a patent's metabolic state of a combination of one or more food items consumed by the patient and one or more activities performed by the patient. For example, a patient may take a diet in combination of performing a physical activity in the same time period. The recommendation module 360 may determine the effect of the patient's metabolic state based on one or more biosignals and classify the food item and/or the physical activity based on the determined effect of the patient's metabolic state using the techniques described above. In one example, a combination of food item and a physical activity determines the overall energy balance of the body. If energy intake from food exceeds energy expenditure from physical activity, excess calories may be stored as fat, leading to weight gain and alterations in metabolic health. Conversely, a negative energy balance, where energy expenditure exceeds intake, can result in weight loss. Therefore, by measuring the weight and monitoring the change of the weight of a patient, the recommendation module 360 may classify the corresponding food item and physical activity accordingly. In another example, a physical activity, such as resistance training, stimulates muscle protein synthesis and increases muscle mass. Since muscle tissue is metabolically active, having a higher proportion of lean muscle mass can elevate resting metabolic rate and enhance overall metabolic health. Additionally, combining a nutrient-rich diet with regular physical activity supports muscle maintenance and growth, contributing to improved metabolic function. In this case, the recommendation module 360 may classify a nutrient-dense food item as low risk/positive health and recommend the food item to the patient.

### V. Additional Considerations

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of the above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B Is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A method for recommending foods to a patient, the method comprising:
accessing a record of food items previously recorded by the patient, the record of food items comprising a current classification of each of a plurality of food items describing an effect of the food item on a metabolic state of the patient;
retrieving a current metabolic profile of the patient, the current metabolic profile comprising 1) biosignal measurements collected during a current time period by one or more wearable sensors worn by the patient and 2) a current metabolic state of the patient determined for the current time period;
encoding, into a vector representation, the record of food items, the biosignal measurements of the current metabolic profile, and the current metabolic state of the patient;
for each of a plurality of food items of the record of food items,
determining an updated classification of the food item by inputting the vector representation to a patient-specific metabolic food model, wherein the patient-specific metabolic food model is iteratively trained to classify the food item based on a training dataset of previously classified food items labeled with a corresponding effect on a metabolic state of a patient; and
identifying a subset of the record of food items comprising food items for which the updated classification differs from the current classification; and
generating, for display to the patient on a computing device, a notification comprising a graphic representation of the identified subset and a recommendation for the patient to consume food items of the identified subset classified as improving the metabolic state of the patient.

2. The method of claim 1, further comprising:
maintaining the record of food items previously recorded by the patient, wherein maintaining the record of food items comprises:
updating the record of food items with each updated classification predicted by the patient-specific metabolic food model; and
updating the record of food items in response to the patient recording a new food item on the computing device.

3. The method of claim 1, wherein determining the updated classification of the food item comprises:
predicting, by the patient-specific metabolic food model, an effect of the food item on the current metabolic state of the patient; and
determining the updated classification of the food item based on the predicted effect of the food item.

4. The method of claim 3, wherein the biosignal measurements comprise blood glucose measurements collected for the patient during the current time period, and wherein the patient-specific metabolic model predicts the effect of the food item based on the collected blood glucose measurements.

5. The method of claim 1, further comprising:
training the patient-specific metabolic food model using the training dataset, wherein each entry of the training dataset comprises a food item, a metabolic state recorded when the food item was consumed, and a label describing an effect of the food item on the metabolic state and describing a classification of the food item.

6. The method of claim 5, further comprising:
updating the training dataset with updated classifications determined by the patient-specific metabolic model; and
retraining the patient-specific metabolic food model based on the updated training dataset.

7. The method of claim 5, wherein training the patient-specific metabolic model comprises:
determining a set of parameter values based on labels assigned to food items in the training dataset, each parameter value describing a weight associated with biosignal measurements and metabolic states of the training dataset.

8. The method of claim 1, further comprising:
responsive to a triggering condition,
accessing the record of food items and the current metabolic profile of the patient; and
applying the patient-specific metabolic model to a plurality of the food items of the record of food items and the current metabolic model to determine an updated classification of each food item.

9. The method of claim 1, further comprising:
receiving a food item recorded by the patient via the computing device;
responsive to determining that the food item does not exist in the record of food items for the patient,
comparing the current metabolic state of the patient to metabolic states of a population of patients who consumed the food item to identify one or more secondary patients; and
determining a baseline classification of the food item for the patient based on classifications of the food item determined for the identified patients.

10. The method of claim 1, wherein the notification comprises a graphic representation of the identified subsets displaying each food item of the identified subset with a color representing the classification of the food item.

11. A method for recommending activities to a patient, the method comprising:
accessing a record of activities previously recorded by a patient, each of a plurality of entries in the record of activities comprising 1) a duration of the activity and 2) biosignal measurements collected for the patient during the activity;
retrieving a current metabolic profile of the patient, the current metabolic profile comprising 1) biosignal measurements collected during a preceding time period by one or more wearable sensors worn by the patient and 2) a metabolic state of the patient determined for the preceding time period;
encoding, into a vector representation, the record of activities, the biosignal measurements of the current metabolic profile, and the current metabolic state of the patient;
for each activity in the record of activities,
determining an effect of the activity on the metabolic state of the patient by inputting the vector representation to a patient-specific metabolic activity model, wherein the metabolic model is iteratively trained to predict the effect of the activity based on a training dataset of previously recorded activities labeled with a corresponding effect on a metabolic state of the patient; and
identifying a subset of activities that improve the metabolic state of the patient based on the effects determined by the patient-specific metabolic model; and
generating, for display to the patient on a computing device, a notification comprising a graphic representation of the identified subset and a recommendation for the patient to perform activities of the identified subset.

12. The method of claim 11, further comprising:
maintaining the record of activities previously recorded by the patient, wherein maintaining the record of activities comprises:
updating the record of activities with the effect of the activity predicted by the patient-specific metabolic model; and
updating the record of activities in response to the patient recording a new activity on the computing device.

13. The method of claim 11, wherein determining the effect of the activity on the metabolic state of the patient comprises:
predicting, by the patient-specific metabolic activity model, the effect of the activity on the metabolic state of the patient; and
determining a classification of the activity based on the predicted effect of the activity, wherein the classification describes whether the activity improves the metabolic state of the patient.

14. The method of claim 13, wherein the biosignal measurements comprise blood glucose measurements collected for the patient during the current time period and wherein the patient-specific metabolic model predicts the effect of the activity based on the collected blood glucose measurements.

15. The method of claim 11, further comprising:
training the patient-specific metabolic model using the training dataset, wherein each entry of the training dataset comprises an activity, a metabolic state recorded when the activity was performed, and a label describing an effect of the activity on the metabolic state.

16. The method of claim 15, wherein the training dataset comprises entries recorded for one or more of:
the patient during one or more time periods preceding the current time period; and
a population of patients during one or more time periods preceding the current time period.

17. The method of claim 15, further comprising:
updating the training dataset with activities recorded by the patient and effects determined by the patient-specific metabolic model; and
responsive to a triggering event, retraining the patient-specific metabolic activity model based on the updated training dataset.

18. The method of claim 15, wherein training the patient-specific metabolic model comprises:
determining a set of parameter values based on labels assigned to activities in the training dataset, each parameter value describing a weight associated with biosignal measurements and metabolic states of the training dataset.

19. The method of claim 11, further comprising:
responsive to a triggering event,
accessing the record of activities and the current metabolic model of the patient; and
applying the patient-specific metabolic model to a plurality of activities of the record of activities and the current metabolic model to determine an updated effect of each activity.

20. The method of claim 11, wherein the training dataset further comprises a time when each previously recorded activity was performed, the method further comprising:
identifying, for each activity in the record of activities, a time that the activity was performed by the patient;
updating the vector representation with a time when each activity of the record of activities was performed by the patient; and
for each activity in the record of activities, determining the effect of the activity when performed at one or more times by inputting the updated vector representation to a patient-specific timing model, wherein the patient-specific timing model is iteratively trained to predict the effect of the activity performed at the one or more times based on the training dataset.

21. A system comprising:
one or more computer processors; and
one or more computer-readable mediums storing instructions that, when executed by the one or more computer processors, cause the system to:
access a record of food items previously recorded by the patient, the record of food items comprising a current classification of each of a plurality of food items describing an effect of the food item on a metabolic state of the patient;
retrieve a current metabolic profile of the patient, the current metabolic profile comprising 1) biosignal measurements collected during a current time period by one or more wearable sensors worn by the patient and 2) a current metabolic state of the patient determined for the current time period;
encode, into a vector representation, the record of food items, the biosignal measurements of the current metabolic profile, and the current metabolic state of the patient;
for each of a plurality of food items of the record of food items,
determine an updated classification of the food item by inputting the vector representation to a patient-specific metabolic food model, wherein the patient-specific metabolic food model is iteratively trained to classify the food item based on a training dataset of previously classified food items labeled with a corresponding effect on a metabolic state of a patient; and
identify a subset of the record of food items comprising food items for which the updated classification differs from the current classification; and
generate, for display to the patient on a computing device, a notification comprising a graphic representation of the identified subset and a recommendation for the patient to consume food items of the identified subset classified as improving the metabolic state of the patient.

22. A non-transitory computer readable storage medium comprising stored instructions that when executed by one or more processors of one or more computing devices, cause the one or more computing devices to:
access a record of food items previously recorded by the patient, the record of food items comprising a current classification of each of a plurality of food items describing an effect of the food item on a metabolic state of the patient;
retrieve a current metabolic profile of the patient, the current metabolic profile comprising 1) biosignal measurements collected during a current time period by one or more wearable sensors worn by the patient and 2) a current metabolic state of the patient determined for the current time period;
encode, into a vector representation, the record of food items, the biosignal measurements of the current metabolic profile, and the current metabolic state of the patient;
for each of a plurality of food items of the record of food items,
determine an updated classification of the food item by inputting the vector representation to a patient-specific metabolic food model, wherein the patient-specific metabolic food model is iteratively trained to classify the food item based on a training dataset of previously classified food items labeled with a corresponding effect on a metabolic state of a patient; and
identify a subset of the record of food items comprising food items for which the updated classification differs from the current classification; and
generate, for display to the patient on a computing device, a notification comprising a graphic representation of the identified subset and a recommendation for the patient to consume food items of the identified subset classified as improving the metabolic state of the patient.

23. A system comprising:
one or more computer processors; and
one or more computer-readable mediums storing instructions that, when executed by the one or more computer processors, cause the system to:
access a record of activities previously recorded by a patient, each of a plurality of entries in the record of activities comprising 1) a duration of the activity and 2) biosignal measurements collected for the patient during the activity;
retrieve a current metabolic profile of the patient, the current metabolic profile comprising 1) biosignal measurements collected during a preceding time period by one or more wearable sensors worn by the patient and 2) a metabolic state of the patient determined for the preceding time period;
encode, into a vector representation, the record of activities, the biosignal measurements of the current metabolic profile, and the current metabolic state of the patient;
for each activity in the record of activities,
determine an effect of the activity on the metabolic state of the patient by inputting the vector representation to a patient-specific metabolic activity model, wherein the metabolic model is iteratively trained to predict the effect of the activity based on a training dataset of previously recorded activities labeled with a corresponding effect on a metabolic state of the patient; and
identify a subset of activities that improve the metabolic state of the patient based on the effects determined by the patient-specific metabolic model; and
generate, for display to the patient on a computing device, a notification comprising a graphic representation of the identified subset and a recommendation for the patient to perform activities of the identified subset.

24. A non-transitory computer readable storage medium comprising stored instructions that when executed by one or more processors of one or more computing devices, cause the one or more computing devices to:
access a record of activities previously recorded by a patient, each of a plurality of entries in the record of activities comprising 1) a duration of the activity and 2) biosignal measurements collected for the patient during the activity;
retrieve a current metabolic profile of the patient, the current metabolic profile comprising 1) biosignal measurements collected during a preceding time period by one or more wearable sensors worn by the patient and 2) a metabolic state of the patient determined for the preceding time period;
encode, into a vector representation, the record of activities, the biosignal measurements of the current metabolic profile, and the current metabolic state of the patient;
for each activity in the record of activities,
determine an effect of the activity on the metabolic state of the patient by inputting the vector representation to a patient-specific metabolic activity model, wherein the metabolic model is iteratively trained to predict the effect of the activity based on a training dataset of previously recorded activities labeled with a corresponding effect on a metabolic state of the patient; and
identify a subset of activities that improve the metabolic state of the patient based on the effects determined by the patient-specific metabolic model; and
generate, for display to the patient on a computing device, a notification comprising a graphic representation of the identified subset and a recommendation for the patient to perform activities of the identified subset.
